SEQUENCE LISTING (101 sequences)

SEQ ID NO: 1 - DNA, 36 bp, Artificial Sequence
  Synthetic
gcacgtagtataatcaactttgaaaaactgtaataa

SEQ ID NO: 2 - DNA, 36 bp, Artificial Sequence
  Synthetic
gcacgttctattatcaacttcgaaaaactataataa

SEQ ID NO: 3 - DNA, 36 bp, Artificial Sequence
  Synthetic
gcccgcagtattatcaatttcgaaaaattataataa

SEQ ID NO: 4 - DNA, 36 bp, Artificial Sequence
  Synthetic
gcgcgctctataattaacttcgaaaaactttaataa

SEQ ID NO: 5 - DNA, 36 bp, Artificial Sequence
  Synthetic
gcacgctccattattaactttgaaaaactttaataa

SEQ ID NO: 6 - DNA, 36 bp, Artificial Sequence
  Synthetic
gctcgctctatcatcaatttcgaaaaactttaataa

SEQ ID NO: 7 - DNA, 36 bp, Artificial Sequence
  Synthetic
gcacgtagtattattaacttcgaaaagttataataa

SEQ ID NO: 8 - DNA, 36 bp, Artificial Sequence
  Synthetic
gcacgttccatcattaactttgaaaaactataataa

SEQ ID NO: 9 - DNA, 36 bp, Artificial Sequence
  Synthetic
gctcgctcaatcatcaactttgaaaagctataataa

SEQ ID NO: 10 - DNA, 36 bp, Artificial Sequence
  Synthetic
gctcgctctatcatcaacttcgaaaaattgtaataa

SEQ ID NO: 11 - DNA, 36 bp, Artificial Sequence
  Synthetic
gctcgctctattatcaattttgaaaaattataataa

SEQ ID NO: 12 - DNA, 36 bp, Artificial Sequence
  Synthetic
gctcgtagtattattaatttcgaaaaattataataa

SEQ ID NO: 13 - DNA, 36 bp, Artificial Sequence
  Synthetic
gctcgttcgattatcaacttcgaaaaactgtaataa

SEQ ID NO: 14 - DNA, 36 bp, Artificial Sequence
  Synthetic
gcaagaagcatcatcaacttcgaaaaactgtaataa

SEQ ID NO: 15 - DNA, 36 bp, Artificial Sequence
  Synthetic
gcgcgttctattattaattttgaaaaattataataa

SEQ ID NO: 16 - protein, 10 aa, Artificial Sequence
  Synthetic
ARSIINFEKL

SEQ ID NO: 17 - DNA, 66 bp, Artificial Sequence
  Synthetic
gattataaagatcatgacggagactataaagaccatgacattgattacaaagacgacgatgacaaa

SEQ ID NO: 18 - DNA, 66 bp, Artificial Sequence
  Synthetic
gactataaagaccacgatggcgattataaagaccatgatattgactacaaagatgatgatgataag

SEQ ID NO: 19 - DNA, 66 bp, Artificial Sequence
  Synthetic
gattataaagatcatgatggcgactataaagatcatgatatcgattacaaagatgacgatgacaaa

SEQ ID NO: 20 - DNA, 66 bp, Artificial Sequence
  Synthetic
gactacaaagatcacgatggtgactacaaagatcacgacattgattataaagacgatgatgacaaa

SEQ ID NO: 21 - DNA, 66 bp, Artificial Sequence
  Synthetic
gattacaaagatcacgatggtgattataaggatcacgatattgattacaaagacgacgacgataaa

SEQ ID NO: 22 - DNA, 66 bp, Artificial Sequence
  Synthetic
gattacaaagatcacgatggcgattacaaagatcatgacattgactacaaagacgatgatgataaa

SEQ ID NO: 23 - DNA, 66 bp, Artificial Sequence
  Synthetic
gattacaaggatcatgatggtgattacaaagatcacgatatcgactacaaagatgatgacgataaa

SEQ ID NO: 24 - DNA, 66 bp, Artificial Sequence
  Synthetic
gactacaaagatcatgatggtgattacaaagatcatgacattgattataaagatgatgatgacaaa

SEQ ID NO: 25 - DNA, 66 bp, Artificial Sequence
  Synthetic
gattataaagaccatgatggtgattataaggatcatgatatcgattataaggatgacgacgataaa

SEQ ID NO: 26 - DNA, 66 bp, Artificial Sequence
  Synthetic
gattataaagatcacgatggcgattataaagaccacgatattgattataaagacgacgatgacaaa

SEQ ID NO: 27 - DNA, 66 bp, Artificial Sequence
  Synthetic
gactataaagaccacgatggtgattataaagatcacgacatcgactacaaagacgatgatgataaa

SEQ ID NO: 28 - DNA, 66 bp, Artificial Sequence
  Synthetic
gactacaaagatcacgacggcgattataaagatcacgatattgactataaagatgacgatgataaa

SEQ ID NO: 29 - DNA, 66 bp, Artificial Sequence
  Synthetic
gattataaagaccatgatggagattacaaagatcatgatattgactataaagacgacgacgataaa

SEQ ID NO: 30 - DNA, 66 bp, Artificial Sequence
  Synthetic
gattataaagatcacgatggtgactacaaagatcacgatatcgattataaagacgatgacgataaa

SEQ ID NO: 31 - DNA, 66 bp, Artificial Sequence
  Synthetic
gactacaaagatcacgatggtgattataaagaccatgatattgattacaaagatgatgatgacaaa

SEQ ID NO: 32 - protein, 22 aa, Artificial Sequence
  Synthetic
DYKDHDGDYKDHDIDYKDDDDK

SEQ ID NO: 33 - protein, 6 aa, Artificial Sequence
  Synthetic
GASGAS

SEQ ID NO: 34 - protein, 6 aa, Artificial Sequence
  Synthetic
GSAGSA

SEQ ID NO: 35 - protein, 4 aa, Artificial Sequence
  Synthetic
GGGG

SEQ ID NO: 36 - protein, 5 aa, Artificial Sequence
  Synthetic
GGGGS

SEQ ID NO: 37 - protein, 8 aa, Artificial Sequence
  Synthetic
VGKGGSGG

SEQ ID NO: 38 - protein, 5 aa, Artificial Sequence
  Synthetic
PAPAP

SEQ ID NO: 39 - protein, 5 aa, Artificial Sequence
  Synthetic
EAAAK

SEQ ID NO: 40 - protein, 6 aa, Artificial Sequence
  Synthetic
AYLAYL

SEQ ID NO: 41 - protein, 6 aa, Artificial Sequence
  Synthetic
LRALRA

SEQ ID NO: 42 - protein, 4 aa, Artificial Sequence
  Synthetic
RLRA

SEQ ID NO: 43 - protein, 32 aa, Artificial Sequence
  Synthetic
KENSISSMAPPASPPASPKTPIEKKHADEIDK

SEQ ID NO: 44 - protein, 19 aa, Artificial Sequence
  Synthetic
KENSISSMAPPASPPASPK

SEQ ID NO: 45 - protein, 14 aa, Artificial Sequence
  Synthetic
KTEEQPSEVNTGPR

SEQ ID NO: 46 - protein, 28 aa, Artificial Sequence
  Synthetic
KESVVDASESDLDSSMQSADESTPQPLK

SEQ ID NO: 47 - protein, 20 aa, Artificial Sequence
  Synthetic
KSEEVNASDFPPPPTDEELR

SEQ ID NO: 48 - protein, 33 aa, Artificial Sequence
  Synthetic
RGGRPTSEEFSSLNSGDFTDDENSETTEEEIDR

SEQ ID NO: 49 - protein, 17 aa, Artificial Sequence
  Synthetic
KQNTASTETTTTNEQPK

SEQ ID NO: 50 - protein, 17 aa, Artificial Sequence
  Synthetic
KQNTANTETTTTNEQPK

SEQ ID NO: 51 - protein, 19 aa, Artificial Sequence
  Synthetic
RSEVTISPAETPESPPATP

SEQ ID NO: 52 - protein, 28 aa, Artificial Sequence
  Synthetic
KASVTDTSEGDLDSSMQSADESTPQPLK

SEQ ID NO: 53 - protein, 20 aa, Artificial Sequence
  Synthetic
KNEEVNASDFPPPPTDEELR

SEQ ID NO: 54 - protein, 33 aa, Artificial Sequence
  Synthetic
RGGIPTSEEFSSLNSGDFTDDENSETTEEEIDR

SEQ ID NO: 55 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 56 - protein, 529 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 57 - protein, 441 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 58 - protein, 416 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 59 - protein, 441 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 60 - DNA, 1323 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 61 - protein, 633 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 62 - protein, 639 aa, Artificial Sequence
  Synthetic
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 63 - protein, 93 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 64 - protein, 200 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 65 - protein, 303 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 66 - protein, 370 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 67 - protein, 390 aa, Artificial Sequence
  Synthetic
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 68 - DNA, 1170 bp, Artificial Sequence
  Synthetic
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 69 - protein, 100 aa, Artificial Sequence
  Synthetic
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 70 - protein, 390 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 71 - DNA, 1170 bp, Artificial Sequence
  Synthetic
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 72 - protein, 226 aa, Artificial Sequence
  Synthetic
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 73 - protein, 5 aa, Artificial Sequence
  Synthetic
QDNKR

SEQ ID NO: 74 - protein, 11 aa, Artificial Sequence
  Synthetic
ECTGLAWEWWR

SEQ ID NO: 75 - protein, 11 aa, Artificial Sequence
  Synthetic
ESLLMWITQCR

SEQ ID NO: 76 - protein, 368 aa, Artificial Sequence
  Synthetic
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 77 - protein, 289 aa, Artificial Sequence
  Synthetic
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 78 - DNA, 1107 bp, Artificial Sequence
  Synthetic
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 79 - DNA, 870 bp, Artificial Sequence
  Synthetic
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 80 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 81 - DNA, 714 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa

SEQ ID NO: 82 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNVFSINGKLGSICGQLLILTYVYGKETPDGIKITLDNLTMQELGYSSGIAHSSAVSRIISKLKQEKVIVYKNSCFYVQNRDYLKRYAPKLDEWFYLACPATWGKLN

SEQ ID NO: 83 - DNA, 713 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatctgattatctcaaaagatatgcccctaaattagatgaatggttttatttagcatgtcctgctacttggggaaaattaaattaa

SEQ ID NO: 84 - DNA, 12 bp, Artificial Sequence
  Synthetic
ggtggtggagga

SEQ ID NO: 85 - DNA, 12 bp, Artificial Sequence
  Synthetic
ggtggaggtgga

SEQ ID NO: 86 - DNA, 12 bp, Artificial Sequence
  Synthetic
ggtggaggaggt

SEQ ID NO: 87 - DNA, 12 bp, Artificial Sequence
  Synthetic
ggaggtggtgga

SEQ ID NO: 88 - DNA, 12 bp, Artificial Sequence
  Synthetic
ggaggaggtggt

SEQ ID NO: 89 - DNA, 12 bp, Artificial Sequence
  Synthetic
ggaggtggaggt

SEQ ID NO: 90 - DNA, 12 bp, Artificial Sequence
  Synthetic
ggaggaggaggt

SEQ ID NO: 91 - DNA, 12 bp, Artificial Sequence
  Synthetic
ggaggaggtgga

SEQ ID NO: 92 - DNA, 12 bp, Artificial Sequence
  Synthetic
ggaggtggagga

SEQ ID NO: 93 - DNA, 12 bp, Artificial Sequence
  Synthetic
ggtggaggagga

SEQ ID NO: 94 - DNA, 12 bp, Artificial Sequence
  Synthetic
ggaggaggagga

SEQ ID NO: 95 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKEATGLAWEAARTVIDDRNLPLVKNRNISIWGTTLYPKYSNKVDNPIE

SEQ ID NO: 96 - protein, 11 aa, Artificial Sequence
  Synthetic
EATGLAWEAAR

SEQ ID NO: 97 - protein, 25 aa, Artificial Sequence
  Synthetic
MKKIMLVFITLILVSLPIAQQTEAK

SEQ ID NO: 98 - protein, 29 aa, Artificial Sequence
  Synthetic
MGLNRFMRAMMVVFITANCITINPDIIFA

SEQ ID NO: 99 - protein, 21 aa, Artificial Sequence
  Synthetic
DYKDHDGDYKDHDIDYKDDDK

SEQ ID NO: 100 - protein, 9 aa, Artificial Sequence
  Synthetic
YMLDLQPET

SEQ ID NO: 101 - protein, 10 aa, Artificial Sequence
  Synthetic
YMLDLQPETT

(19) United States
(12) Patent Application Publication
(10) Pub. No.: US 2021/0239681 A1
(43) Pub. Date: Aug. 5, 2021

(54) COMPOSITIONS AND METHODS FOR EVALUATING POTENCY OF LISTERIA-BASED IMMUNOTHERAPEUTICS

(71) Applicant: ADVAXIS, INC., PRINCETON, NJ (US)

(72) Inventors: Anu WALLECHA, Yardley, PA (US); Mike GRACE, Princeton, NJ (US)

(73) Assignee: ADVAXIS, INC., PRINCETON, NJ (US)

(21) Appl. No.: 17/049,004

(22) PCT Filed: Apr. 25, 2019
(86) PCT No.: PCT/US2019/029066

§ 371 Date: Oct. 19, 2020

Related U.S. Application Data
(60) Provisional application No. 62/663,363, filed on Apr. 27, 2018.

(51) Int. Cl. G01N 33/50

(52) U.S. Cl. CPC G01N 33/505; G01N 2333/555; G01N 2333/195

(57) ABSTRACT

Methods and compositions are provided for assessing antigen presentation and potency of Listeria-based immunotherapeutics in inducing an immune response.

### CROSS-REFERENCE TO RELATED APPLICATIONS

This application claims the benefit of U.S. Application No. 62/663,363, filed Apr. 27, 2018, which is herein incorporated by reference in its entirety for all purposes.

### REFERENCE TO A SEQUENCE LISTING SUBMITTED AS A TEXT FILE VIA EFS WEB

The Sequence Listing written in file 528093_SeqListing_ST25.txt is 89 kilobytes, was created on Feb. 27, 2019, and is hereby incorporated by reference.

### BACKGROUND

Listeria monocytogenes (Lm) is a gram-positive, non-spore forming bacterial organism that is responsible for listeriosis in humans Attenuated Lm strains that can be used to deliver tumor-specific antigen and generate antigen-specific immune response but not cause listeriosis have been bioengineered to provide Lm-based immunotherapies, including cancer immunotherapies. These Lm-based immunotherapeutics utilize the phagosomal escape mechanism to introduce tumor antigens into antigen presenting cells (APCs). Once in the APC, the Lm-based immunotherapeutics express fusion proteins of listeriolysin O (LLO) and a disease-associated or tumor-specific antigen. The antigen is then processed and loaded onto MHC I and MHC II molecules. Presentation of antigen epitopes on MHC II to CD4+T helper cells and in cross-presentation on MHC I to CD8+ cytotoxic T cells, leads to activation of tumor specific T-cell responses.

Previously, to measure the efficacy of Lm-based immunotherapeutic in inducing antigen-specific CD8+ cytotoxic T cells, mice were dosed with the Lm-based immunotherapeutic. Dosing regimens consisted of the administration of a prime dose followed by up to two boost doses of the immunotherapeutic. Once the dosing regimen was completed, and sufficient time was provided for induction of an immune response, animals were sacrificed and spleens removed and processed to assay for the presence and population size of antigen-specific T cells using flow cytometry. This in vivo potency test is time consuming, taking up to a month or more to obtain results, expensive, and not readily amenable to high throughput.

We now describe improved methods for assessing the ability of Listeria-based immunotherapeutics encoding disease-related antigenic peptides to induce activation of CD8+ T cell responses against the antigenic peptide.

### SUMMARY

Methods and compositions are provided for assessing potency of induction of an antigen-specific T-cell response by a Listeria-based immunotherapeutic or potential Listeria-based immunotherapeutic. In some embodiments, the methods comprise: (a) infecting antigen presenting cells (APCs) with the Listeria-based immunotherapeutic or potential Listeria-based immunotherapeutic, wherein the recombinant Listeria-based immunotherapeutic expresses a disease-associated antigenic peptide; (b) co-culturing the infected APCs with T cells having reactivity to the disease-associated antigenic peptide; and (c) determining a cytokine production profile of the T cells, wherein an increase in cytokine production indicates expression of the antigen in infected APCs by the recombinant Listeria-based immunotherapeutic. The described in vitro cell-based assays are faster, less expensive, and more readily amenable to high throughput analyses than previous in vivo assays.

The APCs can be, but are not limited to, monocyte APCs. Monocyte APCs can be, but are not limited to, THP-1 cells. In some embodiments, the T cells having reactivity to the disease-associated antigenic peptide comprises a population of immune cells enriched for T cells having reactivity to the disease-associated antigenic peptide. The cytokine can be, but is not limited to, interferon gamma (IFNγ). Cytokine production can be measured by any method used in the art, including, but not limited to Enzyme-linked immunosorbent assay (ELISA). The disease-associated antigenic peptide can be, but is not limited to, a tumor-associated antigen.

In some embodiments, infecting the APCs comprises incubating the APCs with the Listeria-based immunotherapeutic at a multiplicity of infection (MOI) of about 1 to 200 for 0.5 to 24 hours. In some embodiments, APCs are incubated with the Listeria-based immunotherapeutic for 0.5, 1, 1.5, 2, 3, 4, 5, 6, 7, 8, 9, 10, 12, 14, 16, 18, 20, 22, or 24 hours. In some embodiments, the APCs are incubated with the Listeria-based immunotherapeutic for 0.5-2 hours. In some embodiments, the APCs are incubated with the Listeria-based immunotherapeutic for 2 hours. In some embodiments, after infection, the infected APC cells are washed and cultured for an additional 18-24 hours prior to co-culture with the T cells. In some embodiments, after infection, the infected APCs are washed and cultured for an additional 18, 19, 20, 21, 22, 23, or 24 hours. In some embodiments, after infection, the infected APCs are washed with buffer or media containing gentamycin. In some embodiments, after infection, the infected APCs are incubated with gentamicin for 1-2 hours before washing and incubating for an additional 18-24 hours in the absence of gentamycin.

In some embodiments, the APCs are co-cultured with the T cells for 18-24 hours before determining the cytokine production profile. In some embodiments, the ratio of APC cells to T cells is 1:1 to 4:1. In some embodiments, the number of APC cells is 5000-40,000. In some embodiments, the number of APC cells is 5000, 10000, 15000, 20000, 25000, 30000, 35000, or 40000. In some embodiments the number of T cells is 5000 to 40000. In some embodiments the number of T cells is 5000 to 20,000. In some embodiments, the number of T cells is 5000, 10000, 15000, 20000, 25000, 30000, 35000, or 40000. In some embodiments, the APCs are co-cultured with the T cells in the presence of a protein secretion inhibitor. In some embodiments, the protein secretion inhibitor is brefeldin A.

In some embodiments, the Listeria-based immunotherapeutic comprises a recombinant Listeria strain, wherein the recombinant Listeria strain expresses a disease-associated antigenic peptide. The Listeria-based immunotherapeutic can be, but is not limited to, an L. monocytogenes (Lm)-based immunotherapeutic. In some embodiments, the Listeria-based immunotherapeutic comprises a nucleic acid comprising a first open reading frame encoding a fusion polypeptide, wherein the fusion polypeptide comprises a PEST-containing peptide fused to the disease-associated antigenic peptide. In some embodiments, the PEST-containing peptide is listeriolysin O (LLO) or a fragment thereof. In some embodiments, the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein. In some embodiments, the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO). In some embodiments, the disease-associated antigenic peptide is an antigenic peptide associate with a cancer. In some embodiments, the disease-associated antigenic peptide is a human papillomavirus (HPV) protein E7 or a fragment thereof. In some embodiments, the Lm-based immunotherapeutic is ADXS11-001. ADXS11-001 is a cancer immunotherapy product, which is a live attenuated Listeria monocytogenes strain genetically modified to express a fusion protein of listeriolysin O (LLO) or a fragment thereof and the human papillomavirus (HPV) 16 protein E7 tumor antigen or a fragment thereof. Other suitable Listeria strains are described below.

In some embodiments, the method can comprise: (a) infecting THP-1 cells with a recombinant Listeria-based immunotherapeutic at an MOI of 1-20 for 2 hours to provide infected THP-1 cells, wherein the recombinant Listeria-based immunotherapeutic comprises a live attenuated Listeria monocytogenes strain genetically modified to express a fusion protein of listeriolysin O (LLO) or a fragment thereof and the human papillomavirus (HPV) 16 protein E7 tumor antigen or a fragment thereof; (b) washing the THP-1 cells and culturing the THP-1 cells for an additional 18-24 hours in the absence of gentamicin; (c) co-culturing the infected THP-1 cells with T cells having reactivity to an HPV 16 E7 antigenic peptide for 18-24 hours; and (d) measuring IFNγ production, wherein an increase in IFNγ production indicates expression of the HPV 16 protein E7 tumor antigen in the infected THP-1 cells.

In some embodiments, methods for obtaining a population of enriched antigen-specific T cells having reactivity to a disease-associated antigen are provided. The methods can comprise, for example: a) identifying a peripheral blood mononuclear cell (PBMC) sample having a population of T cells (CD8+ cells) having reactivity to the disease-associated antigen; and b) enriching the population of CD8+ cells having reactivity against the disease-associated antigen. Enriching the population of CD8+ T cells having reactivity against the disease-associated antigen can comprise, for example a) stimulating the cells of the PBMC sample with the disease-associated antigenic peptide; b) identifying and selecting CD8+ cells having reactivity against the disease-associated antigen; c) growing the selected cells; d) restimulating the selected CD8+ cells with the disease-associated antigen; e) identifying and selecting CD8+ cells having reactivity against the disease-associated antigen; and f) repeating steps c-e for 2-10 rounds. The percentage of cells in the sample that are T cells having reactivity against the disease-associated antigen can be enriched to greater that 5%, greater than 10%, greater than 25%, greater than 75%, greater than 80%, greater than 85%, greater than 90%, or greater than 95%. Identifying and selecting CD8+ cells having reactivity against the disease-associated antigen can be done by methods in the art including, but no limited to, flow cytometry and fluorescence-activated cell sorting (FACS).

### BRIEF DESCRIPTION OF THE FIGURES

FIG. 1. Bar graph illustrating presentation of E7 epitope in the presence of gentamicin.

FIG. 2. Bar graph illustrating presentation of E7 Epitope in the absence of gentamicin.

FIGS. 3A-3B. FACS analysis of intracellular cytokine, IFNγ, staining in THP-1 cells following stimulation with control antibody (FIG. 3A) or 9mer (FIG. 3B).

FIG. 3C. FACS analysis of intracellular cytokine, IFNγ, staining in THP-1 cells following stimulation with 10mer.

FIG. 4. Illustration of the general process for Listeria strain ADXS11-001.

FIG. 5. FACS analysis of YMLDLQPETT-specific CD8+ T cells in donor 224 following culture with 9mer (A) or 10mer (2T) (B).

FIG. 6. FACS analysis of YMLDLQPETT-specific CD8+ T cells from donor 224 following stimulation with negative control (A) or 10mer (2T) peptide (B).

FIG. 7. FACS analysis of YMLDLQPETT-specific CD8+ T cells following 0 (primary), and 1, 2, or 3 rounds of restimulation with 10mer.

FIG. 8. FACS analysis showing YMLDLQPETT-specific CD8+ T cells in primary culture (A) or after multiple rounds of restimulation (B).

FIG. 9. FACS analysis showing IFNγ detection in T cells after stimulation with control antibody (A), 9mer (B), and 10mer (C).

FIG. 10. FACS analysis showing basal IFNγ detection in THP-1 cells after stimulation with control antibody (A), 9mer (B), and 10mer (C).

FIG. 11. Graph illustrating peptide titration with E7 specific T cells.

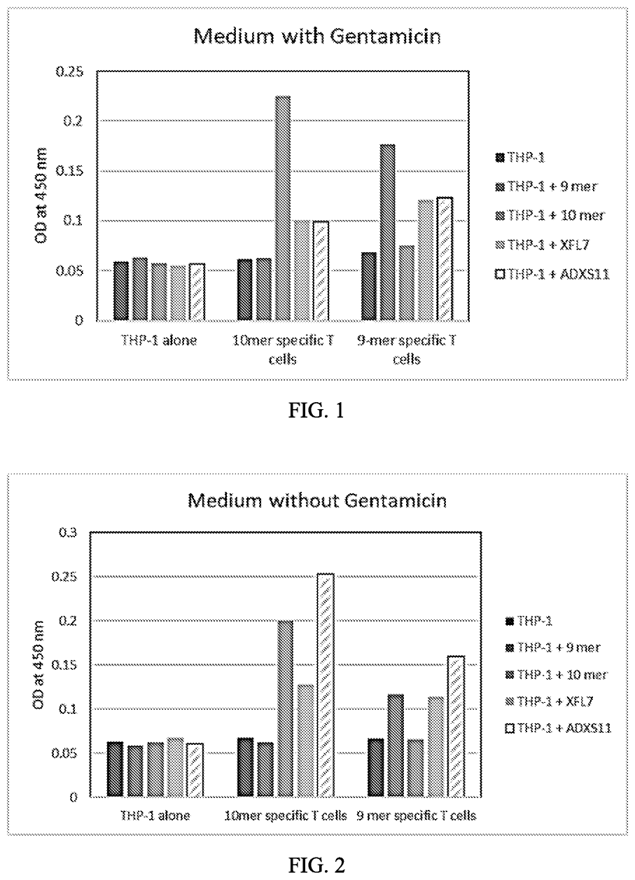

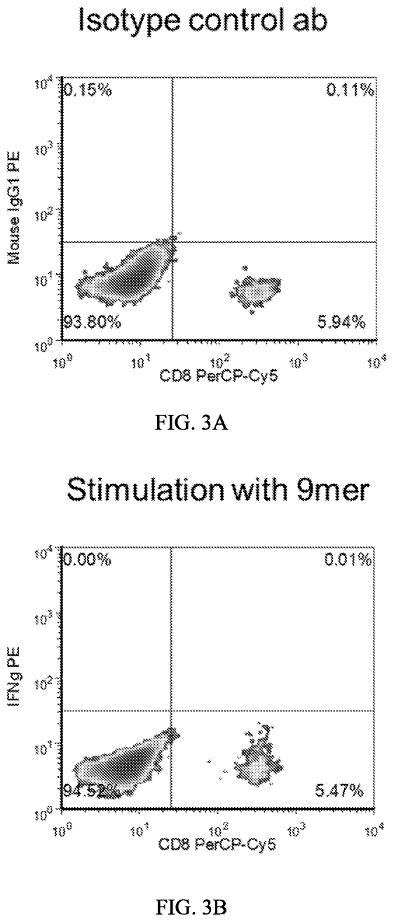

### DEFINITIONS

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The term “fusion protein” refers to a protein comprising two or more peptides linked together by peptide bonds or other chemical bonds. The peptides can be linked together directly by a peptide or other chemical bond. For example, a chimeric molecule can be recombinantly expressed as a single-chain fusion protein. Alternatively, the peptides can be linked together by a “linker” such as one or more amino acids or another suitable linker between the two or more peptides.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given Listeria cell or any other host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “plasmid” or “vector” includes any known delivery vector including a bacterial delivery vector, a viral vector delivery vector, a peptide immunotherapy delivery vector, a DNA immunotherapy delivery vector, an episomal plasmid, an integrative plasmid, or a phage vector. The term “vector” refers to a construct which is capable of delivering, and, optionally, expressing, one or more fusion polypeptides in a host cell.

The term “episomal plasmid” or “extrachromosomal plasmid” refers to a nucleic acid vector that is physically separate from chromosomal DNA (i.e., episomal or extrachromosomal and does not integrated into a host cell's genome) and replicates independently of chromosomal DNA. A plasmid may be linear or circular, and it may be single-stranded or double-stranded. Episomal plasmids may optionally persist in multiple copies in a host cell's cytoplasm (e.g., Listeria), resulting in amplification of any genes of interest within the episomal plasmid.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “stably maintained” refers to maintenance of a nucleic acid molecule or plasmid in the absence of selection (e.g., antibiotic selection) for at least 10 generations without detectable loss. For example, the period can be at least 15 generations, 20 generations, at least 25 generations, at least 30 generations, at least 40 generations, at least 50 generations, at least 60 generations, at least 80 generations, at least 100 generations, at least 150 generations, at least 200 generations, at least 300 generations, or at least 500 generations. Stably maintained can refer to a nucleic acid molecule or plasmid being maintained stably in cells in vitro (e.g., in culture), being maintained stably in vivo, or both.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “wild type” refers to entities having a structure and/or activity as found in a normal (as contrasted with mutant, diseased, altered, or so forth) state or context. Wild type gene and polypeptides often exist in multiple different forms (e.g., alleles).

The term “isolated” with respect to proteins and nucleic acid refers to proteins and nucleic acids that are relatively purified with respect to other bacterial, viral or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

“Exogenous” or “heterologous” molecules or sequences are molecules or sequences that are not normally expressed in a cell or are not normally present in a cell in that form. Normal presence includes presence with respect to the particular developmental stage and environmental conditions of the cell. An exogenous or heterologous molecule or sequence, for example, can include a mutated version of a corresponding endogenous sequence within the cell or can include a sequence corresponding to an endogenous sequence within the cell but in a different form (i.e., not within a chromosome). An exogenous or heterologous molecule or sequence in a particular cell can also be a molecule or sequence derived from a different species than a reference species of the cell or from a different organism within the same species. For example, in the case of a Listeria strain expressing a heterologous polypeptide, the heterologous polypeptide could be a polypeptide that is not native or endogenous to the Listeria strain, that is not normally expressed by the Listeria strain, from a source other than the Listeria strain, derived from a different organism within the same species.

In contrast, “endogenous” molecules or sequences or “native” molecules or sequences are molecules or sequences that are normally present in that form in a particular cell at a particular developmental stage under particular environmental conditions.

The term “variant” refers to an amino acid or nucleic acid sequence (or an organism or tissue) that is different from the majority of the population but is still sufficiently similar to the common mode to be considered to be one of them (e.g., splice variants).

The term “isoform” refers to a version of a molecule (e.g., a protein) with only slight differences compared to another isoform, or version (e.g., of the same protein). For example, protein isoforms may be produced from different but related genes, they may arise from the same gene by alternative splicing, or they may arise from single nucleotide polymorphisms.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “analog” when referring to a protein means a protein that differs from a naturally occurring protein by conservative amino acid differences, by modifications which do not affect amino acid sequence, or by both.

The term “functional” refers to the innate ability of a protein or nucleic acid (or a fragment, isoform, or variant thereof) to exhibit a biological activity or function. Such biological activities or functions can include, for example, the ability to elicit an immune response when administered to a subject. Such biological activities or functions can also include, for example, binding to an interaction partner. In the case of functional fragments, isoforms, or variants, these biological functions may in fact be changed (e.g., with respect to their specificity or selectivity), but with retention of the basic biological function.

The terms “immunogenicity” or “immunogenic” refer to the innate ability of a molecule (e.g., a protein, a nucleic acid, an antigen, or an organism) to elicit an immune response in a subject when administered to the subject Immunogenicity can be measured, for example, by a greater number of antibodies to the molecule, a greater diversity of antibodies to the molecule, a greater number of T cells specific for the molecule, a greater cytotoxic or helper T-cell response to the molecule, and the like.

The term “antigen” is used herein to refer to a substance that, when placed in contact with a subject or organism (e.g., when present in or when detected by the subject or organism), results in a detectable immune response from the subject or organism. An antigen may be, for example, a lipid, a protein, a carbohydrate, a nucleic acid, or combinations and variations thereof. For example, an “antigenic peptide” refers to a peptide that leads to the mounting of an immune response in a subject or organism when present in or detected by the subject or organism. For example, such an “antigenic peptide” may encompass proteins that are loaded onto and presented on MHC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The term “epitope” refers to a site on an antigen that is recognized by the immune system (e.g., to which an antibody binds). An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “mutation” refers to the any change of the structure of a gene or a protein. For example, a mutation can result from a deletion, an insertion, a substitution, or a rearrangement of chromosome or a protein. An “insertion” changes the number of nucleotides in a gene or the number of amino acids in a protein by adding one or more additional nucleotides or amino acids. A “deletion” changes the number of nucleotides in a gene or the number of amino acids in a protein by reducing one or more additional nucleotides or amino acids.

A “frameshift” mutation in DNA occurs when the addition or loss of nucleotides changes a gene's reading frame. A reading frame consists of groups of 3 bases that each code for one amino acid. A frameshift mutation shifts the grouping of these bases and changes the code for amino acids. The resulting protein is usually nonfunctional. Insertions and deletions can each be frameshift mutations.

A “missense” mutation or substitution refers to a change in one amino acid of a protein or a point mutation in a single nucleotide resulting in a change in an encoded amino acid. A point mutation in a single nucleotide that results in a change in one amino acid is a “nonsynonymous” substitution in the DNA sequence. Nonsynonymous substitutions can also result in a “nonsense” mutation in which a codon is changed to a premature stop codon that results in truncation of the resulting protein. In contrast, a “synonymous” mutation in a DNA is one that does not alter the amino acid sequence of a protein (due to codon degeneracy).

The term “somatic mutation” includes genetic alterations acquired by a cell other than a germ cell (e.g., sperm or egg). Such mutations can be passed on to progeny of the mutated cell in the course of cell division but are not inheritable. In contrast, a germinal mutation occurs in the germ line and can be passed on to the next generation of offspring.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations ±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p<0.05.

### DETAILED DESCRIPTION

### I. Overview

Listeria-based immunotherapeutics infect APCs and express fusion proteins containing disease-associated antigens. The disease-associated antigens are then processed and loaded onto MHC by the APC and presented to T cells, leading to activation of an immune response. Disclosed are in vitro cell-based assays for determining potency of a Listeria-based immunotherapeutic in inducing a T cell response to a disease-associated antigenic antigen. In some embodiments, the methods comprise: (a) infecting antigen presenting cells (APCs) in culture with the Listeria-based immunotherapeutic or potential Listeria-based immunotherapeutic, wherein the recombinant Listeria-based immunotherapeutic expresses a disease-associated antigenic peptide; (b) co-culturing the infected APCs with T cells having reactivity to the disease-associated antigenic peptide; and (c) determining a cytokine production profile of the T cells, wherein an increase in cytokine production indicates expression of the disease-associated antigen in infected APCs by the Listeria-based immunotherapeutic and effective presentation to T cells.

The described methods can be used, for example, to evaluate antigen presentation or induction of an immune response by a Listeria-based immunotherapeutics or to assess potency or infectivity of a Listeria-based immunotherapeutic.

As one specific example, ADXS11-001 is a recombinant Listeria monocytogenes (Lm) strain attenuated due to the irreversible deletion of prfA in the genome and, further, its complementation with mutated prfA gene (D133V). The prfA gene regulates the transcription of several virulence genes such as hly (Listeriolysin O or LLO), actA (Actin nucleator A), plcA (phospholipase A), and plcB (phospholipase B), that are required for in vivo intracellular growth and survival of Lm. The complementation with mutated prfA in ADXS11-001 causes a reduction in the expression of the virulence genes. The plasmid in the ADXS11-001 immunotherapeutic also contains a disease-associated antigenic peptide human papillomavirus protein E7 fused to truncated Listeriolysin O (tLLO)) under the control of the hly promoter. In order to evaluate induction of E7 antigen presentation in APCs infected with ADXS11-001, THP-1 cells are infected in vitro with ADXS11-001. The infected THP-1 cells are then co-cultured with T cells known to be reactive to E7 antigen. Presentation of the E7 antigen on the THP-1 cells results in activation of the T cells, leading to interferon gamma (IFNγ) cytokine production by the T cells. Thus potency of ADXS11-001 in stimulating E7 antigen presentation by the THP-1 cells can be measured by monitoring IFNγ levels produced by E7 responsive T cells co-cultured with the infected THP-1 cells.

The biological activity of ADXS11-001 relies upon uptake of ADXS11-001 by antigen presenting cells (APC) such as macrophages and dendritic cells, its escape from phagolysosome, intracellular replication in the cytosol of APC, expression of tLLO-E7, processing, and presentation of tLLO-E7 on surface of APC to stimulate E7-specific cytotoxic T cell response. Using THP-1 cells in combination with E7 responsive T cells to determine potency is a superior alternative to infecting mice with ADXS11-001, waiting several weeks for the mice to develop an immune response, isolating T cells from spleen and examining the profile of the isolated T cells to determine the level of T cell specific immune response to E7 antigen. The described methods are advantageous in providing a reliable, quantitative, in vitro method of assessing Lm-based immunotherapeutic function that is faster and more economical than in vivo testing using mice.

### II. Methods for Evaluating Potency of Listeria and Antigen Presentation In Vitro

Disclosed herein are methods for evaluating potency of Listeria-based immunotherapeutics and antigen presentation in an in vitro cell-based assay. In some embodiments, the methods comprise: (a) infecting antigen presenting cells (APCs) in culture with the Listeria-based immunotherapeutic or potential Listeria-based immunotherapeutic, wherein the recombinant Listeria-based immunotherapeutic expresses a disease-associated antigenic peptide; (b) co-culturing the infected APCs with T cells having reactivity to the disease-associated antigenic peptide; and (c) determining a cytokine production profile of the T cells, wherein an increase in cytokine production indicates expression of the disease-associated antigen in infected APCs by the Listeria-based immunotherapeutic and effective presentation to T cells.

In some embodiments, a cell-based assay for evaluating potency of Listeria-based immunotherapeutic can comprise:
a) preparing a culture of actively dividing APCs,
b) infecting the actively dividing APCs with a Listeria-based immunotherapeutic expressing a disease-associated antigenic peptide,
c) washing the APCs of step b) and culturing the washed APCs in growth media,
d) collecting the APCs of step c) and co-culturing with T cells reactive to the disease-associated antigenic peptide, and
e) determining a level of cytokine production compared to cytokine production in control samples.

In some embodiments the APC is a THP-1 cell.

In some embodiments, the disease-associated antigenic peptide is a tumor-associated antigen. A tumor-associated antigen can be, but is not limited to, an HPV E7 antigen.

In some embodiments, the T cells having reactivity to the disease-associated antigenic peptide comprises a population of immune cells enriched for T cells having reactivity to the disease-associated antigenic peptide. In some embodiments, the percent of disease-associated antigenic peptide-specific T cells in the T cell population is at least 5%, at least 10%, at least 25%, at least 50%, at least 75%, at least 90%, or at least 95%. In some embodiments, the T cells are enriched in E7 peptide-specific T cells. In some embodiments, the T cells are enriched in 1T-peptide-specific T cells. In some embodiments, the T cells are enriched in 2T-peptide-specific T cells.

The cytokine can be, but is not limited to, interferon gamma (IFNγ). Cytokine production can be measured by any method used in the art, including, but not limited to enzyme-linked immunosorbent assay (ELISA). In some embodiments, secreted cytokines are measured. In some embodiments, intracellular cytokine levels are measured.

In some embodiments, the APCs are infected with the Listeria-based immunotherapeutic at a multiplicity of infection (MOI) of 1 to 200. In some embodiments, the MOI is about 1, about 2, about 5, about 10, about 20, about 100, or about 200. In some embodiments, the MOI is about 1-50, 1-40, 1-30, 1-20, 1-10, or 1-5. In some embodiments, MOI is about 1, 2, 3, 4, 5, 6, 7, 8, 9, 10, 11, 12, 13, 14, 15, 16, 17, 18, 19, 20, 30, 40, 50, 75, 100, 125, 150, 175, or 200.

In some embodiments, the APCs are exposed to the Listeria-based immunotherapeutic for 0.5 to 24 hours. In some embodiments, the APCs are exposed to the Listeria-based immunotherapeutic for about 0.5, 1, 1.5, 2, 3, 4, 5, 6, 7, 8, 10, 12, 14, 16, 18, 20, 22, or 24 hours.

In some embodiments, the APC cells are infected for 0.5-2 hours, washed to remove extracellular bacteria and further cultured In some embodiments, after infection, the infected APC cells are washed and cultured for an additional 18-24 hours prior to co-culture with the T cells. In some embodiments, after infection, the infected APCs are washed and cultured for an additional 18, 19, 20, 21, 22, 23, or 24 hours prior to co-culture with the T cells. In some embodiments, the APCs are cultured in the absence of an antibiotic. In some embodiments, the APCs are cultured in the absence of gentamicin. In some embodiments, after infection, the infected APCs are washed with buffer or media containing gentamycin. In some embodiments, after infection, the infected APCs are incubated with gentamicin for 1-2 hours before washing and incubating for an additional 18-24 hours in the absence of gentamycin. Culturing the APCs prior to co-culture with T cells provides time to the Listeria-based immunotherapeutic to express the disease-based antigenic peptide and for the APC to process and present the antigenic peptide.

In some embodiments, the APCs are co-cultured with the T cells for 18-24 hours before determining the cytokine production. In some embodiments, the APCs are co-cultured with the T cells in the presence of a protein secretion inhibitor, such as brefeldin A. In some embodiments the T cells comprise a population of T cells enriched in T cells reactive with the disease-associated antigenic peptide. The percentage of cells in the sample that are T cells having reactivity against the disease-associated antigen can be enriched to greater that 5%, greater than 10%, greater than 25%, greater than 75%, greater than 80%, greater than 85%, greater than 90%, or greater than 95%. In some embodiments the T cells are reactive against HPV E7, peptide 1T (SEQ ID NO: 100) and/or peptide 2T (SEQ ID NO: 101).

In some embodiments, the ratio of APC cells to T cells is 1:1 to 4:1. In some embodiments, the number of APC cells is 5000-40,000. In some embodiments, the number of APC cells is 5000, 10000, 15000, 20000, 25000, 30000, 35000, or 40000. In some embodiments the number of T cells is 5000 to 40000. In some embodiments the number of T cells is 5000 to 40000. In some embodiments the number of T cells is 5000 to 20,000. In some embodiments, the number of T cells is 5000, 10000, 15000, 20000, 25000, 30000, 35000, or 40000.

In some embodiments, the cytokine is IFNγ. Induction of cytokine production resulting from infection by the Listeria-based immunotherapeutic is determined by comparing the level of cytokine production in the sample with control samples, including, but not limited to, APCs infected with Listeria not expressing the disease-associate antigenic peptide or expressing a different disease-associate antigenic peptide or uninfected APCs.

In some embodiments, the Listeria-based immunotherapeutic comprises a recombinant Listeria strain, wherein the recombinant Listeria strain expresses a disease-associated antigenic peptide. The Listeria-based immunotherapeutic can be, but is not limited to, an L. monocytogenes (Lm)-based immunotherapeutic. In some embodiments, the Listeria-based immunotherapeutic comprises a nucleic acid comprising a first open reading frame encoding a fusion polypeptide, wherein the fusion polypeptide comprises a PEST-containing peptide fused to the disease-associated antigenic peptide. In some embodiments, the PEST-containing peptide is listeriolysin O (LLO) or a fragment thereof. In some embodiments, the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein. In some embodiments, the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO). In some embodiments, the disease-associated antigenic peptide is an antigenic peptide associate with a cancer. In some embodiments, the disease-associated antigenic peptide is a human papillomavirus (HPV) protein E7 or a fragment thereof. In some embodiments, the Lm-based immunotherapeutic is ADXS11-001. ADXS11-001 is a cancer immunotherapy product, which is a live attenuated Listeria monocytogenes strain genetically modified to express a fusion protein of listeriolysin O (LLO) and the human papillomavirus (HPV) 16 protein E7 tumor antigen.

In some embodiments, a cell-based assay for evaluating potency of Listeria-based immunotherapeutic comprises:
a) preparing a culture of actively dividing THP-1 cells,
b) infecting the actively dividing THP-1 cells with a Listeria-based immunotherapeutic expressing a disease-associated antigenic peptide at an MOI of 1-200 for 1-24 hours,
c) washing the infected THP-1 cells and culturing the washed infected THP-1 cells in growth media without gentamycin for 18-24 hours,
d) collecting about 5000-40,000 THP-1 cells from step c) and co-culturing with T cells reactive to the disease-associated antigenic peptide at ratio of 1:1 to 1:4 (T cells to THP-1 cells) for about 18 to about 24 hours, and
e) determining a level of INFγ production compared to INFγ production in control samples.

In some embodiments, the described cell-based assays provide for quantitative determination of epitope presentation in THP1 cells infected with a Listeria-based immunotherapeutic or potential Listeria-based immunotherapeutic. The described in vitro cell-based assays are faster, less expensive, and more readily amenable to high throughput analyses then previous in vivo assays.

Methods and compositions are provided for assessing potency of and/or antigen presentation in by recombinant bacteria or Listeria strains, such as Listeria monocytogenes. Examples of recombinant Listeria strains that can be used in such methods are provided in more detail elsewhere herein. Such methods utilize macrophage cell lines or macrophage-like cell lines with macrophage phenotypes. Such cells can be immortalized cells. For example, the cell line can be a human monocyte cell line such as THP-1 cells. THP-1 designates a spontaneously immortalized monocyte-like cell line, derived from the peripheral blood of a childhood case of acute monocytic leukemia (M5 subtype). THP-1 cells can be differentiated into macrophage-like cells using, for example, phorbol 12-myristate 13-acetate (commonly known as PMA or TPA).

Additional embodiments are disclosed in the examples.

### III. Recombinant Bacteria or Listeria Strains

The methods disclosed herein assess potency of and antigen presentation by bacteria strains, such as a Listeria strain (i.e., a Listeria-based immunotherapeutic). Such bacteria strains can be recombinant bacteria strains. Such recombinant bacteria strains can comprise a recombinant fusion polypeptide disclosed herein or a nucleic acid encoding the recombinant fusion polypeptide as disclosed elsewhere herein. In some embodiments, the bacteria strain is a Listeria strain, such as a Listeria monocytogenes (Lm) strain. Lm has a number of inherent advantages as a vaccine vector. The bacterium grows very efficiently in vitro without special requirements, and it lacks LPS, which is a major toxicity factor in gram-negative bacteria, such as Salmonella. Genetically attenuated Lm vectors also offer additional safety as they can be readily eliminated with antibiotics, in case of serious adverse effects, and unlike some viral vectors, no integration of genetic material into the host genome occurs.

The recombinant Listeria strain can be any Listeria strain. Examples of suitable Listeria strains include Listeria seeligeri, Listeria grayi, Listeria ivanovii, Listeria murrayi, Listeria welshimeri, Listeria monocytogenes (Lm), or any other known Listeria species. In some embodiments, the recombinant Listeria strain is a strain of the species Listeria monocytogenes. Examples of Listeria monocytogenes strains include the following: L. monocytogenes 10403S wild type (see, e.g., Bishop and Hinrichs (1987) J Immunol 139:2005-2009; Lauer et al. (2002) J Bact 184:4177-4186); L. monocytogenes DP-L4056, which is phage cured (see, e.g., Lauer et al. (2002) J Bact 184:4177-4186); L. monocytogenes DP-L4027, which is phage cured and has an hly gene deletion (see, e.g., Lauer et al. (2002) J Bact 184:4177-4186; Jones and Portnoy (1994) Infect Immunity 65:5608-5613); L. monocytogenes DP-L4029, which is phage cured and has an actA gene deletion (see, e.g., Lauer et al. (2002) J Bact 184:4177-4186; Skoble et al. (2000) J Cell Biol 150:527-538); L. monocytogenes DP-L4042 (delta PEST) (see, e.g., Brockstedt et al. (2004) Proc Natl Acad Sci. USA 101:13832-13837 and supporting information); L. monocytogenes DP-L4097 (LLO-S44A) (see, e.g., Brockstedt et al. (2004) Proc Natl Acad Sci USA 101:13832-13837 and supporting information); L. monocytogenes DP-L4364 (delta lplA; lipoate protein ligase) (see, e.g., Brockstedt et al. (2004) Proc Natl Acad Sci USA 101:13832-13837 and supporting information); L. monocytogenes DP-L4405 (delta inlA) (see, e.g., Brockstedt et al. (2004) Proc Natl Acad Sci USA 101:13832-13837 and supporting information); L. monocytogenes DP-L4406 (delta inlB) (see, e.g., Brockstedt et al. (2004) Proc Natl Acad Sci USA 101:13832-13837 and supporting information); L. monocytogenes CS-L0001 (delta actA; delta inlB) (see, e.g., Brockstedt et al. (2004) Proc Natl Acad Sci USA 101:13832-13837 and supporting information); L. monocytogenes CS-L0002 (delta actA; delta lplA) (see, e.g., Brockstedt et al. (2004) Proc Natl Acad Sci USA 101:13832-13837 and supporting information); L monocytogenes CS-L0003 (LLO L461T; delta lplA) (see, e.g., Brockstedt et al. (2004) Proc Natl Acad Sci USA 101:13832-13837 and supporting information); L. monocytogenes DP-L4038 (delta actA; LLO L461T) (see, e.g., Brockstedt et al. (2004) Proc Natl Acad Sci USA 101:13832-13837 and supporting information); L. monocytogenes DP-L4384 (LLO S44A; LLO L461T) (see, e.g., Brockstedt et al. (2004) Proc Natl Acad Sci USA 101:13832-13837 and supporting information); a L. monocytogenes strain with an lplA1 deletion (encoding lipoate protein ligase LplA1) (see, e.g., O'Riordan et al. (2003) Science 302:462-464); L. monocytogenes DP-L4017 (10403S with LLO L461T) (see, e.g., U.S. Pat. No. 7,691,393); L. monocytogenes EGD (see, e.g., GenBank Accession No. AL591824). In some embodiments, the Listeria strain is L. monocytogenes EGD-e (see GenBank Accession No. NC_003210; ATCC Accession No. BAA-679); L. monocytogenes DP-L4029 (actA deletion, optionally in combination with uvrAB deletion (DP-L4029uvrAB) (see, e.g., U.S. Pat. No. 7,691,393); L. monocytogenes actA-linlB—double mutant (see, e.g., ATCC Accession No. PTA-5562); L. monocytogenes lplA mutant or hly mutant (see, e.g., US 2004/0013690); L. monocytogenes dalldat double mutant (see, e.g., US 2005/0048081). Other L. monocytogenes strains includes those that are modified (e.g., by a plasmid and/or by genomic integration) to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, or uptake by a host cell. Each of the above references is herein incorporated by reference in its entirety for all purposes.

The recombinant bacteria or Listeria can have wild-type virulence, can have attenuated virulence, or can be avirulent. For example, a recombinant Listeria of can be sufficiently virulent to escape the phagosome or phagolysosome and enter the cytosol. Such Listeria strains can also be live-attenuated Listeria strains, which comprise at least one attenuating mutation, deletion, or inactivation as disclosed elsewhere herein. In some embodiments, the recombinant Listeria is an attenuated auxotrophic strain. An auxotrophic strain is one that is unable to synthesize a particular organic compound required for its growth. Examples of such strains are described in U.S. Pat. No. 8,114,414, herein incorporated by reference in its entirety for all purposes.

In some embodiments, the recombinant Listeria strain lacks antibiotic resistance genes. For example, such recombinant Listeria strains can comprise a plasmid that does not encode an antibiotic resistance gene. However, some recombinant Listeria strains provided herein comprise a plasmid comprising a nucleic acid encoding an antibiotic resistance gene. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin.

A. Bacteria or Listeria Strains Comprising Recombinant Fusion Polypeptides or Nucleic Acids Encoding Recombinant Fusion Polypeptides

The recombinant bacteria strains (e.g., Listeria strains) disclosed herein comprise a recombinant fusion polypeptide disclosed herein or a nucleic acid encoding the recombinant fusion polypeptide as disclosed elsewhere herein.

In bacteria or Listeria strains comprising a nucleic acid encoding a recombinant fusion protein, the nucleic acid can be codon optimized. Examples of optimal codons utilized by L. monocytogenes for each amino acid are shown US 2007/0207170, herein incorporated by reference in its entirety for all purposes. A nucleic acid is codon-optimized if at least one codon in the nucleic acid is replaced with a codon that is more frequently used by L. monocytogenes for that amino acid than the codon in the original sequence.

The nucleic acid can be present in an episomal plasmid within the bacteria or Listeria strain and/or the nucleic acid can be genomically integrated in the bacteria or Listeria strain. Some recombinant bacteria or Listeria strains comprise two separate nucleic acids encoding two recombinant fusion polypeptides as disclosed herein: one nucleic acid in an episomal plasmid, and one genomically integrated in the bacteria or Listeria strain.

The episomal plasmid can be one that is stably maintained in vitro (in cell culture), in vivo (in a host), or both in vitro and in vivo. If in an episomal plasmid, the open reading frame encoding the recombinant fusion polypeptide can be operably linked to a promoter/regulatory sequence in the plasmid. If genomically integrated in the bacteria or Listeria strain, the open reading frame encoding the recombinant fusion polypeptide can be operably linked to an exogenous promoter/regulatory sequence or to an endogenous promoter/regulatory sequence. Examples of promoters/regulatory sequences useful for driving constitutive expression of a gene are well-known and include, for example, an hly, hlyA, actA, prfA, and p60 promoters of Listeria, the Streptococcus bac promoter, the Streptomyces griseus sgiA promoter, and the B. thuringiensis phaZ promoter. In some cases, an inserted gene of interest is not interrupted or subjected to regulatory constraints which often occur from integration into genomic DNA, and in some cases, the presence of the inserted heterologous gene does not lead to rearrangement or interruption of the cell's own important regions.

Such recombinant bacteria or Listeria strains can be made by transforming a bacteria or Listeria strain or an attenuated bacteria or Listeria strain described elsewhere herein with a plasmid or vector comprising a nucleic acid encoding the recombinant fusion polypeptide. The plasmid can be an episomal plasmid that does not integrate into a host chromosome. Alternatively, the plasmid can be an integrative plasmid that integrates into a chromosome of the bacteria or Listeria strain. The plasmids used herein can also be multicopy plasmids. Methods for transforming bacteria are well-known, and include calcium-chloride competent cell-based methods, electroporation methods, bacteriophage-mediated transduction, chemical transformation techniques, and physical transformation techniques. See, e.g., de Boer et al. (1989) Cell 56:641-649; Miller et al. (1995) FASEB J. 9:190-199; Sambrook et al. (1989) Molecular Cloning: A Laboratory Manual, Cold Spring Harbor Laboratory, New York; Ausubel et al. (1997) Current Protocols in Molecular Biology, John Wiley & Sons, New York; Gerhardt et al., eds., 1994, Methods for General and Molecular Bacteriology, American Society for Microbiology, Washington, D.C.; and Miller, 1992, A Short Course in Bacterial Genetics, Cold Spring Harbor Laboratory Press, Cold Spring Harbor, N.Y., each of which is herein incorporated by reference in its entirety for all purposes.

Bacteria or Listeria strains with genomically integrated heterologous nucleic acids can be made, for example, by using a site-specific integration vector, whereby the bacteria or Listeria comprising the integrated gene is created using homologous recombination. The integration vector can be any site-specific integration vector that is capable of infecting a bacteria or Listeria strain. Such an integration vector can comprise, for example, a PSA attPP′ site, a gene encoding a PSA integrase, a U153 attPP′ site, a gene encoding a U153 integrase, an A118 attPP′ site, a gene encoding an A118 integrase, or any other known attPP′ site or any other phage integrase.

Such bacteria or Listeria strains comprising an integrated gene can also be created using any other known method for integrating a heterologous nucleic acid into a bacteria or Listeria chromosome. Techniques for homologous recombination are well-known, and are described, for example, in Baloglu et al. (2005) Vet Microbiol 109(1-2):11-17); Jiang et al. 2005) Acta Biochim Biophys Sin (Shanghai) 37(1):19-24), and U.S. Pat. No. 6,855,320, each of which is herein incorporated by reference in its entirety for all purposes.

Integration into a bacteria or Listerial chromosome can also be achieved using transposon insertion. Techniques for transposon insertion are well-known, and are described, for example, for the construction of DP-L967 by Sun et al. (1990) Infection and Immunity 58: 3770-3778, herein incorporated by reference in its entirety for all purposes. Transposon mutagenesis can achieve stable genomic insertion, but the position in the genome where the heterologous nucleic acids has been inserted is unknown.

Integration into a bacterial or Listerial chromosome can also be achieved using phage integration sites (see, e.g., Lauer et al. (2002) J Bacteriol 184(15):4177-4186, herein incorporated by reference in its entirety for all purposes). For example, an integrase gene and attachment site of a bacteriophage (e.g., U153 or PSA listeriophage) can be used to insert a heterologous gene into the corresponding attachment site, which may be any appropriate site in the genome (e.g. comK or the 3′ end of the arg tRNA gene). Endogenous prophages can be cured from the utilized attachment site prior to integration of the heterologous nucleic acid. Such methods can result, for example, in single-copy integrants. In order to avoid a “phage curing step,” a phage integration system based on PSA phage can be used (see, e.g., Lauer et al. (2002) J Bacteriol 184:4177-4186, herein incorporated by reference in its entirety for all purposes). Maintaining the integrated gene can require, for example, continuous selection by antibiotics. Alternatively, a phage-based chromosomal integration system can be established that does not require selection with antibiotics. Instead, an auxotrophic host strain can be complemented. For example, a phage-based chromosomal integration system for clinical applications can be used, where a host strain that is auxotrophic for essential enzymes, including, for example, D-alanine racemase is used (e.g., Lm dal(−)dat(−)).

Conjugation can also be used to introduce genetic material and/or plasmids into bacteria. Methods for conjugation are well-known, and are described, for example, in Nikodinovic et al. (2006) Plasmid 56(3):223-227 and Auchtung et al. (2005) Proc Natl Acad Sci USA 102(35):12554-12559, each of which is herein incorporated by reference in its entirety for all purposes.

In a specific example, a recombinant bacteria or Listeria strain can comprise a nucleic acid encoding a recombinant fusion polypeptide genomically integrated into the bacteria or Listeria genome as an open reading frame with an endogenous actA sequence (encoding an ActA protein) or an endogenous hly sequence (encoding an LLO protein). For example, the expression and secretion of the fusion polypeptide can be under the control of the endogenous actA promoter and ActA signal sequence or can be under the control of the endogenous hly promoter and LLO signal sequence. As another example, the nucleic acid encoding a recombinant fusion polypeptide can replace an actA sequence encoding an ActA protein or an hly sequence encoding an LLO protein.

Selection of recombinant bacteria or Listeria strains can be achieved by any means. For example, antibiotic selection can be used. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin. Alternatively, auxotrophic strains can be used, and an exogenous metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. Alternatively, a temperature-sensitive plasmid can be used to select recombinants or any other known means for selecting recombinants.

B. Attenuation of Bacteria or Listeria Strains

The recombinant bacteria strains (e.g., recombinant Listeria strains) disclosed herein can be attenuated. The term “attenuation” encompasses a diminution in the ability of the bacterium to cause disease in a host animal. For example, the pathogenic characteristics of an attenuated Listeria strain may be lessened compared with wild-type Listeria, although the attenuated Listeria is capable of growth and maintenance in culture. Using as an example the intravenous inoculation of BALB/c mice with an attenuated Listeria, in some embodiments, the lethal dose at which 50% of inoculated animals survive (LD₅₀) is increased above the LD₅₀ of wild-type Listeria by at least about 10-fold, at least about 100-fold, at least about 1,000-fold, at least about 10,000-fold, or at least about 100,000-fold. An attenuated strain of Listeria is thus one that does not kill an animal to which it is administered, or is one that kills the animal only when the number of bacteria administered is vastly greater than the number of wild-type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. Attenuated strains are environmentally safe in that they are incapable of uncontrolled replication.

(I) Methods of Attenuating Bacteria and Listeria Strains

Attenuation can be accomplished by any known means. For example, such attenuated strains can be deficient in one or more endogenous virulence genes or one or more endogenous metabolic genes. Examples of such genes are disclosed herein, and attenuation can be achieved by inactivation of any one of or any combination of the genes disclosed herein. Inactivation can be achieved, for example, through deletion or through mutation (e.g., an inactivating mutation). The term “mutation” includes any type of mutation or modification to the sequence (nucleic acid or amino acid sequence) and may encompass a deletion, a truncation, an insertion, a substitution, a disruption, or a translocation. For example, a mutation can include a frameshift mutation, a mutation which causes premature termination of a protein, or a mutation of regulatory sequences which affect gene expression. Mutagenesis can be accomplished using recombinant DNA techniques or using traditional mutagenesis technology using mutagenic chemicals or radiation and subsequent selection of mutants. Deletion mutants may be preferred because of the accompanying low probability of reversion. The term “metabolic gene” refers to a gene encoding an enzyme involved in or required for synthesis of a nutrient utilized or required by a host bacteria. For example, the enzyme can be involved in or required for the synthesis of a nutrient required for sustained growth of the host bacteria. The term “virulence” gene includes a gene whose presence or activity in an organism's genome that contributes to the pathogenicity of the organism (e.g., enabling the organism to achieve colonization of a niche in the host (including attachment to cells), immunoevasion (evasion of host's immune response), immunosuppression (inhibition of host's immune response), entry into and exit out of cells, or obtaining nutrition from the host).

A specific example of such an attenuated strain is Listeria monocytogenes (Lm) dal(−)dat(−) (Lmdd). Another example of such an attenuated strain is Lm dal(−)dat(−)AactA (LmddA). See, e.g., US 2011/0142791, herein incorporated by references in its entirety for all purposes. LmddA is based on a Listeria strain which is attenuated due to the deletion of the endogenous virulence gene actA. Such strains can retain a plasmid for antigen expression in vivo and in vitro by complementation of the dal gene. Alternatively, the LmddA can be a dal/dat/actA Listeria having mutations in the endogenous dal, dat, and actA genes. Such mutations can be, for example, a deletion or other inactivating mutation.

Another specific example of an attenuated strain is Lm prfA(−) or a strain having a partial deletion or inactivating mutation in the prfA gene. The PrfA protein controls the expression of a regulon comprising essential virulence genes required by Lm to colonize its vertebrate hosts; hence the prfA mutation strongly impairs PrfA ability to activate expression of PrfA-dependent virulence genes.

Yet another specific example of an attenuated strain is Lm inlB(−)actA(−) in which two genes critical to the bacterium's natural virulence—internalin B and act A—are deleted.

Other examples of attenuated bacteria or Listeria strains include bacteria or Listeria strains deficient in one or more endogenous virulence genes. Examples of such genes include actA, prfA, plcB, plcA, inlA, inlB, inlC, inlJ, and bsh in Listeria. Attenuated Listeria strains can also be the double mutant or triple mutant of any of the above-mentioned strains. Attenuated Listeria strains can comprise a mutation or deletion of each one of the genes, or comprise a mutation or deletion of, for example, up to ten of any of the genes provided herein (e.g., including the actA, prfA, and dal/dat genes). For example, an attenuated Listeria strain can comprise a mutation or deletion of an endogenous internalin C (inlC) gene and/or a mutation or deletion of an endogenous actA gene. Alternatively, an attenuated Listeria strain can comprise a mutation or deletion of an endogenous internalin B (inlB) gene and/or a mutation or deletion of an endogenous actA gene. Alternatively, an attenuated Listeria strain can comprise a mutation or deletion of endogenous inlB, inlC, and actA genes. Translocation of Listeria to adjacent cells is inhibited by the deletion of the endogenous actA gene and/or the endogenous inlC gene or endogenous inlB gene, which are involved in the process, thereby resulting in high levels of attenuation with increased immunogenicity and utility as a strain backbone. An attenuated Listeria strain can also be a double mutant comprising mutations or deletions of both plcA and plcB. In some cases, the strain can be constructed from the EGD Listeria backbone.

A bacteria or Listeria strain can also be an auxotrophic strain having a mutation in a metabolic gene. As one example, the strain can be deficient in one or more endogenous amino acid metabolism genes. For example, the generation of auxotrophic strains of Listeria deficient in D-alanine, for example, may be accomplished in a number of ways that are well-known, including deletion mutations, insertion mutations, frameshift mutations, mutations which cause premature termination of a protein, or mutation of regulatory sequences which affect gene expression. Deletion mutants may be preferred because of the accompanying low probability of reversion of the auxotrophic phenotype. As an example, mutants of D-alanine which are generated according to the protocols presented herein may be tested for the ability to grow in the absence of D-alanine in a simple laboratory culture assay. Those mutants which are unable to grow in the absence of this compound can be selected.

Examples of endogenous amino acid metabolism genes include a vitamin synthesis gene, a gene encoding pantothenic acid synthase, a D-glutamic acid synthase gene, a D-alanine amino transferase (dat) gene, a D-alanine racemase (dal) gene, dga, a gene involved in the synthesis of diaminopimelic acid (DAP), a gene involved in the synthesis of Cysteine synthase A (cysK), a vitamin-B12 independent methionine synthase, trpA, trpB, trpE, asnB, gltD, gltB, leuA, argG, and thrC. The Listeria strain can be deficient in two or more such genes (e.g., dat and dal). D-glutamic acid synthesis is controlled in part by the dal gene, which is involved in the conversion of D-glu+pyr to alpha-ketoglutarate+D-ala, and the reverse reaction.

As another example, an attenuated Listeria strain can be deficient in an endogenous synthase gene, such as an amino acid synthesis gene. Examples of such genes include folP, a gene encoding a dihydrouridine synthase family protein, ispD, ispF, a gene encoding a phosphoenolpyruvate synthase, hisF, hisH, fliI, a gene encoding a ribosomal large subunit pseudouridine synthase, ispD, a gene encoding a bifunctional GMP synthase/glutamine amidotransferase protein, cobS, cobB, cbiD, a gene encoding a uroporphyrin-III C-methyltransferase/uroporphyrinogen-III synthase, cob Q, uppS, truB, dxs, mvaS, dapA, ispG, folC, a gene encoding a citrate synthase, argJ, a gene encoding a 3-deoxy-7-phosphoheptulonate synthase, a gene encoding an indole-3-glycerol-phosphate synthase, a gene encoding an anthranilate synthase/glutamine amidotransferase component, menB, a gene encoding a menaquinone-specific isochorismate synthase, a gene encoding a phosphoribosylformylglycinamidine synthase I or II, a gene encoding a phosphoribosylaminoimidazole-succinocarboxamide synthase, carB, carA, thyA, mgsA, aroB, hepB, rluB, ilvB, ilvN, alsS, fabF, fabH, a gene encoding a pseudouridine synthase, pyrG, truA, pabB, and an atp synthase gene (e.g., atpC, atpD-2, aptG, atpA-2, and so forth).

Attenuated Listeria strains can be deficient in endogenous phoP, aroA, aroC, aroD, or plcB. As yet another example, an attenuated Listeria strain can be deficient in an endogenous peptide transporter. Examples include genes encoding an ABC transporter/ATP-binding/permease protein, an oligopeptide ABC transporter/oligopeptide-binding protein, an oligopeptide ABC transporter/permease protein, a zinc ABC transporter/zinc-binding protein, a sugar ABC transporter, a phosphate transporter, a ZIP zinc transporter, a drug resistance transporter of the EmrB/QacA family, a sulfate transporter, a proton-dependent oligopeptide transporter, a magnesium transporter, a formate/nitrite transporter, a spermidine/putrescine ABC transporter, a Na/Pi-cotransporter, a sugar phosphate transporter, a glutamine ABC transporter, a major facilitator family transporter, a glycine betaine/L-proline ABC transporter, a molybdenum ABC transporter, a techoic acid ABC transporter, a cobalt ABC transporter, an ammonium transporter, an amino acid ABC transporter, a cell division ABC transporter, a manganese ABC transporter, an iron compound ABC transporter, a maltose/maltodextrin ABC transporter, a drug resistance transporter of the Bcr/CflA family, and a subunit of one of the above proteins.

Other attenuated bacteria and Listeria strains can be deficient in an endogenous metabolic enzyme that metabolizes an amino acid that is used for a bacterial growth process, a replication process, cell wall synthesis, protein synthesis, metabolism of a fatty acid, or for any other growth or replication process. Likewise, an attenuated strain can be deficient in an endogenous metabolic enzyme that can catalyze the formation of an amino acid used in cell wall synthesis, can catalyze the synthesis of an amino acid used in cell wall synthesis, or can be involved in synthesis of an amino acid used in cell wall synthesis. Alternatively, the amino acid can be used in cell wall biogenesis. Alternatively, the metabolic enzyme is a synthetic enzyme for D-glutamic acid, a cell wall component.

Other attenuated Listeria strains can be deficient in metabolic enzymes encoded by a D-glutamic acid synthesis gene, dga, an alr (alanine racemase) gene, or any other enzymes that are involved in alanine synthesis. Yet other examples of metabolic enzymes for which the Listeria strain can be deficient include enzymes encoded by serC (a phosphoserine aminotransferase), asd (aspartate betasemialdehyde dehydrogenase; involved in synthesis of the cell wall constituent diaminopimelic acid), the gene encoding gsaB-glutamate-1-semialdehyde aminotransferase (catalyzes the formation of 5-aminolevulinate from (S)-4-amino-5-oxopentanoate), hemL (catalyzes the formation of 5-aminolevulinate from (S)-4-amino-5-oxopentanoate), aspB (an aspartate aminotransferase that catalyzes the formation of oxalozcetate and L-glutamate from L-aspartate and 2-oxoglutarate), argF-1 (involved in arginine biosynthesis), aroE (involved in amino acid biosynthesis), aroB (involved in 3-dehydroquinate biosynthesis), aroD (involved in amino acid biosynthesis), aroC (involved in amino acid biosynthesis), hisB (involved in histidine biosynthesis), hisD (involved in histidine biosynthesis), hisG (involved in histidine biosynthesis), metX (involved in methionine biosynthesis), proB (involved in proline biosynthesis), argR (involved in arginine biosynthesis), argJ (involved in arginine biosynthesis), thil (involved in thiamine biosynthesis), LMOf2365_1652 (involved in tryptophan biosynthesis), aroA (involved in tryptophan biosynthesis), ilvD (involved in valine and isoleucine biosynthesis), ilvC (involved in valine and isoleucine biosynthesis), leuA (involved in leucine biosynthesis), dapF (involved in lysine biosynthesis), and thrB (involved in threonine biosynthesis) (all GenBank Accession No. NC_002973).

An attenuated Listeria strain can be generated by mutation of other metabolic enzymes, such as a tRNA synthetase. For example, the metabolic enzyme can be encoded by the trpS gene, encoding tryptophanyl tRNA synthetase. For example, the host strain bacteria can be Δ(trpS aroA), and both markers can be contained in an integration vector.

Other examples of metabolic enzymes that can be mutated to generate an attenuated Listeria strain include an enzyme encoded by murE (involved in synthesis of diaminopimelic acid; GenBank Accession No: NC_003485), LMOf2365_2494 (involved in teichoic acid biosynthesis), WecE (Lipopolysaccharide biosynthesis protein rffA; GenBank Accession No: AE014075.1), or amiA (an N-acetylmuramoyl-L-alanine amidase). Yet other examples of metabolic enzymes include aspartate aminotransferase, histidinol-phosphate aminotransferase (GenBank Accession No. NP_466347), or the cell wall teichoic acid glycosylation protein GtcA.

Other examples of metabolic enzymes that can be mutated to generate an attenuated Listeria strain include a synthetic enzyme for a peptidoglycan component or precursor. The component can be, for example, UDP-N-acetylmuramylpentapeptide, UDP-N-acetylglucosamine, MurNAc-(pentapeptide)-pyrophosphoryl-undecaprenol, GlcNAc-p-(1,4)-MurNAc-(pentapeptide)-pyrophosphorylundecaprenol, or any other peptidoglycan component or precursor.

Yet other examples of metabolic enzymes that can be mutated to generate an attenuated Listeria strain include metabolic enzymes encoded by murG, murD, murA-1, or murA-2 (all set forth in GenBank Accession No. NC_002973). Alternatively, the metabolic enzyme can be any other synthetic enzyme for a peptidoglycan component or precursor. The metabolic enzyme can also be a trans-glycosylase, a trans-peptidase, a carboxy-peptidase, any other class of metabolic enzyme, or any other metabolic enzyme. For example, the metabolic enzyme can be any other Listeria metabolic enzyme or any other Listeria monocytogenes metabolic enzyme.

Other bacteria strains can be attenuated as described above for Listeria by mutating the corresponding orthologous genes in the other bacteria strains.

(2) Methods of Complementing Attenuated Bacteria and Listeria Strains

The attenuated bacteria or Listeria strains disclosed herein can further comprise a nucleic acid comprising a complementing gene or encoding a metabolic enzyme that complements an attenuating mutation (e.g., complements the auxotrophy of the auxotrophic Listeria strain). For example, a nucleic acid having a first open reading frame encoding a fusion polypeptide as disclosed herein can further comprise a second open reading frame comprising the complementing gene or encoding the complementing metabolic enzyme. Alternatively, a first nucleic acid can encode the fusion polypeptide and a separate second nucleic acid can comprise the complementing gene or encode the complementing metabolic enzyme.

The complementing gene can be extrachromosomal or can be integrated into the bacteria or Listeria genome. For example, the auxotrophic Listeria strain can comprise an episomal plasmid comprising a nucleic acid encoding a metabolic enzyme. Such plasmids will be contained in the Listeria in an episomal or extrachromosomal fashion. Alternatively, the auxotrophic Listeria strain can comprise an integrative plasmid (i.e., integration vector) comprising a nucleic acid encoding a metabolic enzyme. Such integrative plasmids can be used for integration into a Listeria chromosome. In some embodiments, the episomal plasmid or the integrative plasmid lacks an antibiotic resistance marker.

The metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. For example, a bacteria auxotrophic for D-glutamic acid synthesis can be transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. Similarly, a bacterium auxotrophic for D-alanine synthesis will grow in the absence of D-alanine when transformed and expressing a plasmid comprising a nucleic acid encoding an amino acid metabolism enzyme for D-alanine synthesis. Such methods for making appropriate media comprising or lacking necessary growth factors, supplements, amino acids, vitamins, antibiotics, and the like are well-known and are available commercially.

Once the auxotrophic bacteria comprising the plasmid encoding a metabolic enzyme or a complementing gene provided herein have been selected in appropriate medium, the bacteria can be propagated in the presence of a selective pressure. Such propagation can comprise growing the bacteria in media without the auxotrophic factor. The presence of the plasmid expressing the metabolic enzyme or the complementing gene in the auxotrophic bacteria ensures that the plasmid will replicate along with the bacteria, thus continually selecting for bacteria harboring the plasmid. Production of the bacteria or Listeria strain can be readily scaled up by adjusting the volume of the medium in which the auxotrophic bacteria comprising the plasmid are growing.

In one specific example, the attenuated strain is a strain having a deletion of or an inactivating mutation in dal and dat (e.g., Listeria monocytogenes (Lm) dal(−)dat(−) (Lmdd) or Lm dal(−)dat(−)AactA (LmddA)), and the complementing gene encodes an alanine racemase enzyme (e.g., encoded by dal gene) or a D-amino acid aminotransferase enzyme (e.g., encoded by dat gene). An exemplary alanine racemase protein can have the sequence set forth in SEQ ID NO: 76 (encoded by SEQ ID NO: 78; GenBank Accession No: AF038438) or can be a homologue, variant, isoform, analog, fragment, fragment of a homologue, fragment of a variant, fragment of an analog, or fragment of an isoform of SEQ ID NO: 76. The alanine racemase protein can also be any other Listeria alanine racemase protein. Alternatively, the alanine racemase protein can be any other gram-positive alanine racemase protein or any other alanine racemase protein. An exemplary D-amino acid aminotransferase protein can have the sequence set forth in SEQ ID NO: 77 (encoded by SEQ ID NO: 79; GenBank Accession No: AF038439) or can be a homologue, variant, isoform, analog, fragment, fragment of a homologue, fragment of a variant, fragment of an analog, or fragment of an isoform of SEQ ID NO: 77. The D-amino acid aminotransferase protein can also be any other Listeria D-amino acid aminotransferase protein. Alternatively, the D-amino acid aminotransferase protein can be any other gram-positive D-amino acid aminotransferase protein or any other D-amino acid aminotransferase protein.

In another specific example, the attenuated strain is a strain having a deletion of or an inactivating mutation in prfA (e.g., Lm prfA(−)), and the complementing gene encodes a PrfA protein. For example, the complementing gene can encode a mutant PrfA (D133V) protein that restores partial PrfA function. An example of a wild type PrfA protein is set forth in SEQ ID NO: 80 (encoded by nucleic acid set forth in SEQ ID NO: 81), and an example of a D133V mutant PrfA protein is set forth in SEQ ID NO: 82 (encoded by nucleic acid set forth in SEQ ID NO: 83). The complementing PrfA protein can be a homologue, variant, isoform, analog, fragment, fragment of a homologue, fragment of a variant, fragment of an analog, or fragment of an isoform of SEQ ID NO: 80 or 82. The PrfA protein can also be any other Listeria PrfA protein. Alternatively, the PrfA protein can be any other gram-positive PrfA protein or any other PrfA protein.

In another example, the bacteria strain or Listeria strain can comprise a deletion of or an inactivating mutation in an actA gene, and the complementing gene can comprise an actA gene to complement the mutation and restore function to the Listeria strain.

Other auxotroph strains and complementation systems can also be adopted for the use with the methods and compositions provided herein.

### IV. Recombinant Fusion Polypeptides

The recombinant fusion polypeptides in the recombinant bacteria or Listeria strains disclosed herein can be in any form. Some such fusion polypeptides can comprise a PEST-containing peptide fused to one or more disease-associated antigenic peptides. Other such recombinant fusion polypeptides can comprise one or more disease-associated antigenic peptides, and wherein the fusion polypeptide does not comprise a PEST-containing peptide.

Another example of a recombinant fusion polypeptides comprises from N-terminal end to C-terminal end a bacterial secretion sequence, a ubiquitin (Ub) protein, and one or more disease-associated antigenic peptides (i.e., in tandem, such as Ub-peptide1-peptide2). Alternatively, if two or more disease-associated antigenic peptides are used, a combination of separate fusion polypeptides can be used in which each antigenic peptide is fused to its own secretion sequence and Ub protein (e.g., Ub1-peptide1; Ub2-peptide2).

Nucleic acids (termed minigene constructs) encoding such recombinant fusion polypeptides are also disclosed. Such minigene nucleic acid constructs can further comprise two or more open reading frames linked by a Shine-Dalgarno ribosome binding site nucleic acid sequence between each open reading frame. For example, a minigene nucleic acid construct can further comprise two to four open reading frames linked by a Shine-Dalgarno ribosome binding site nucleic acid sequence between each open reading frame. Each open reading frame can encode a different polypeptide. In some nucleic acid constructs, the codon encoding the carboxy terminus of the fusion polypeptide is followed by two stop codons to ensure termination of protein synthesis.

The bacterial signal sequence can be a Listerial signal sequence, such as an Hly or an ActA signal sequence, or any other known signal sequence. In other cases, the signal sequence can be an LLO signal sequence. An exemplary LLO signal sequence is set forth in SEQ ID NO: 97. The signal sequence can be bacterial, can be native to a host bacterium (e.g., Listeria monocytogenes, such as a secA1 signal peptide), or can be foreign to a host bacterium. Specific examples of signal peptides include an Usp45 signal peptide from Lactococcus lactis, a Protective Antigen signal peptide from Bacillus anthracia, a secA2 signal peptide such the p60 signal peptide from Listeria monocytogenes, and a Tat signal peptide such as a B. subtilis Tat signal peptide (e.g., PhoD). In specific examples, the secretion signal sequence is from a Listeria protein, such as an ActA₃₀₀ secretion signal or an ActA₁₀₀ secretion signal. An exemplary ActA signal sequence is set forth in SEQ ID NO: 98.

The ubiquitin can be, for example, a full-length protein. The ubiquitin expressed from the nucleic acid construct provided herein can be cleaved at the carboxy terminus from the rest of the recombinant fusion polypeptide expressed from the nucleic acid construct through the action of hydrolases upon entry to the host cell cytosol. This liberates the amino terminus of the fusion polypeptide, producing a peptide in the host cell cytosol.

Selection of, variations of, and arrangement of antigenic peptides within a fusion polypeptide are discussed in detail elsewhere herein, and examples of disease-associated antigenic peptides are discussed in more detail elsewhere herein.

The recombinant fusion polypeptides can comprise one or more tags. For example, the recombinant fusion polypeptides can comprise one or more peptide tags N-terminal and/or C-terminal to one or more antigenic peptides. A tag can be fused directly to an antigenic peptide or linked to an antigenic peptide via a linker (examples of which are disclosed elsewhere herein). Examples of tags include the following: FLAG tag; 2×FLAG tag; 3×FLAG tag; His tag, 6×His tag; and SIINFEKL tag. An exemplary SIINFEKL tag is set forth in SEQ ID NO: 16 (encoded by any one of the nucleic acids set forth in SEQ ID NOS: 1-15). An exemplary 3×FLAG tag is set forth in SEQ ID NO: 32 (encoded by any one of the nucleic acids set forth in SEQ ID NOS: 17-31). An exemplary variant 3×FLAG tag is set forth in SEQ ID NO: 99. Two or more tags can be used together, such as a 2×FLAG tag and a SIINFEKL tag, a 3×FLAG tag and a SIINFEKL tag, or a 6×His tag and a SIINFEKL tag. If two or more tags are used, they can be located anywhere within the recombinant fusion polypeptide and in any order. For example, the two tags can be at the C-terminus of the recombinant fusion polypeptide, the two tags can be at the N-terminus of the recombinant fusion polypeptide, the two tags can be located internally within the recombinant fusion polypeptide, one tag can be at the C-terminus and one tag at the N-terminus of the recombinant fusion polypeptide, one tag can be at the C-terminus and one internally within the recombinant fusion polypeptide, or one tag can be at the N-terminus and one internally within the recombinant fusion polypeptide. Other tags include chitin binding protein (CBP), maltose binding protein (MBP), glutathione-S-transferase (GST), thioredoxin (TRX), and poly(NANP). Particular recombinant fusion polypeptides comprise a C-terminal SIINFEKL tag. Such tags can allow for easy detection of the recombinant fusion protein, confirmation of secretion of the recombinant fusion protein, or for following the immunogenicity of the secreted fusion polypeptide by following immune responses to these “tag” sequence peptides. Such immune response can be monitored using a number of reagents including, for example, monoclonal antibodies and DNA or RNA probes specific for these tags.

The recombinant fusion polypeptides disclosed herein can be expressed by recombinant Listeria strains or can be expressed and isolated from other vectors and cell systems used for protein expression and isolation. Recombinant Listeria strains comprising expressing such antigenic peptides can be used, for example in immunogenic compositions comprising such recombinant Listeria and in vaccines comprising the recombinant Listeria strain and an adjuvant. Expression of one or more antigenic peptides as a fusion polypeptides with a nonhemolytic truncated form of LLO, ActA, or a PEST-like sequence in host cell systems in Listeria strains and host cell systems other than Listeria can result in enhanced immunogenicity of the antigenic peptides.

Nucleic acids encoding such recombinant fusion polypeptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames (e.g., an open reading frame encoding the recombinant fusion polypeptide and a second open reading frame encoding a metabolic enzyme). In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different polypeptide. In some nucleic acids, the codon encoding the carboxy terminus of the fusion polypeptide is followed by two stop codons to ensure termination of protein synthesis.

A. Antigenic Peptides

Disease-associated peptides include peptides from proteins that are expressed in a particular disease. For example, such peptides may be from proteins that are expressed in a disease tissue but not in a corresponding normal tissue, or that are expressed at abnormally high levels in a disease tissue. The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life. Examples of disease-associated antigenic peptides can include human papillomavirus (HPV) E7 or E6, a Prostate Specific Antigen (PSA), a chimeric Her2 antigen, Her2/neu chimeric antigen. The human papillomavirus can be HPV 16 or HPV 18. The antigenic peptide can also include HPV16 E6, HPV16 E7, HPV18 E6, HPV18 E7 antigens operably linked in tandem or HPV16 antigenic peptide operably linked in tandem to an HPV antigenic peptide.

The fusion polypeptide can include a single antigenic peptide or can includes two or more antigenic peptides. Each antigenic peptide can be of any length sufficient to induce an immune response, and each antigenic peptide can be the same length or the antigenic peptides can have different lengths. For example, an antigenic peptide disclosed herein can be 5-100, 15-50, or 21-27 amino acids in length, or 15-100, 15-95, 15-90, 15-85, 15-80, 15-75, 15-70, 15-65, 15-60, 15-55, 15-50, 15-45, 15-40, 15-35, 15-30, 20-100, 20-95, 20-90, 20-85, 20-80, 20-75, 20-70, 20-65, 20-60, 20-55, 20-50, 20-45, 20-40, 20-35, 20-30, 11-21, 15-21, 21-31, 31-41, 41-51, 51-61, 61-71, 71-81, 81-91, 91-101, 101-121, 121-141, 141-161, 161-181, 181-201, 8-27, 10-30, 10-40, 15-30, 15-40, 15-25, 1-10, 10-20, 20-30, 30-40, 1-100, 5-75, 5-50, 5-40, 5-30, 5-20, 5-15, 5-10, 1-75, 1-50, 1-40, 1-30, 1-20, 1-15, 1-10, 8-11, or 11-16 amino acids in length. For example, an antigenic peptide can be at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids in length. Some specific examples of antigenic peptides are 21 or 27 amino acids in length. Other antigenic peptides can be full-length proteins or fragments thereof.

As one example, an antigenic peptide can comprise a neoepitope. These neoepitopes can be, for example, patient-specific (i.e., subject-specific) cancer mutations. Antigenic peptides comprising neoepitopes can be generated in a process for creating a personalized immunotherapy comprising comparing nucleic acids extracted from a cancer sample from a subject to nucleic acids extracted from a normal or healthy reference sample in order to identify somatic mutations or sequence differences present in the cancer sample compared with the normal or healthy sample. For examples, these mutations or sequence differences can be somatic, nonsynonymous missense mutations, or somatic frameshift mutations, and can encode an expressed amino acid sequence. A peptide expressing such somatic mutations or sequence differences can be referred to as a “neoepitope.” A cancer-specific neoepitope may refer to an epitope that is not present in a reference sample (such as a normal non-cancerous or germline cell or tissue) but is found in a cancer sample. This includes, for example, situations in which in a normal non-cancerous or germline cell a corresponding epitope is found, but due to one or more mutations in a cancer cell, the sequence of the epitope is changed so as to result in the neoepitope. A neoepitope can comprise a mutated epitope, and can comprise non-mutated sequence on either or both sides of the mutation.

As another example, antigenic peptides can comprise recurrent cancer mutations. For example, a recombinant fusion polypeptide disclosed herein can comprise a PEST-containing peptide fused to two or more antigenic peptides (i.e., in tandem, such as PEST-peptide1-peptide2) or can comprise two or more antigenic peptides not fused to a PEST-containing peptide, wherein each antigenic peptide comprises a single, recurrent cancer mutation (i.e., a single, recurrent change in the amino acid sequence of a protein, or a sequence encoded by a single, different, nonsynonymous, recurrent cancer mutation in a gene), and wherein at least two of the antigenic peptides comprise different recurrent cancer mutations and are fragments of the same cancer-associated protein. Alternatively, each of the antigenic peptides can comprise a different recurrent cancer mutation from a different cancer-associated protein. Alternatively, a combination of separate fusion polypeptides can be used in which each antigenic peptide is fused (or is not fused) to its own PEST-containing peptide (e.g., PEST1-peptide1; PEST2-peptide2). Optionally, some or all of the fragments are non-contiguous fragments of the same cancer-associated protein. Non-contiguous fragments are fragments that do not occur sequentially in a protein sequence (e.g., the first fragment consists of residues 10-30, and the second fragment consists of residues 100-120; or the first fragment consists of residues 10-30, and the second fragment consists of residues 20-40). Optionally, each of the antigenic peptides comprises a different recurrent cancer mutation from a single type of cancer.

Recurrent cancer mutations can be from cancer-associated proteins. The term “cancer-associated protein” includes proteins having mutations that occur in multiple types of cancer, that occur in multiple subjects having a particular type of cancer, or that are correlated with the occurrence or progression of one or more types of cancer. For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). In some embodiments, a cancer-associated protein has a “mutational hotspot.” A mutational hotspot is an amino acid position in a protein-coding gene that is mutated (preferably by somatic substitutions rather than other somatic abnormalities, such as translocations, amplifications, and deletions) more frequently than would be expected in the absence of selection. Such hotspot mutations can occur across multiple types of cancer and/or can be shared among multiple cancer patients. Mutational hotspots indicate selective pressure across a population of tumor samples. Tumor genomes contain recurrent cancer mutations that “drive” tumorigenesis by affecting genes (i.e., tumor driver genes) that confer selective growth advantages to the tumor cells upon alteration. Such tumor driver genes can be identified, for example, by identifying genes that are mutated more frequently than expected from the background mutation rate (i.e., recurrence); by identifying genes that exhibit other signals of positive selection across tumor samples (e.g., a high rate of non-silent mutations compared to silent mutations, or a bias towards the accumulation of functional mutations); by exploiting the tendency to sustain mutations in certain regions of the protein sequence based on the knowledge that whereas inactivating mutations are distributed along the sequence of the protein, gain-of-function mutations tend to occur specifically in particular residues or domains; or by exploiting the overrepresentation of mutations in specific functional residues, such as phosphorylation sites. Many of these mutations frequently occur in the functional regions of biologically active proteins (for example, kinase domains or binding domains) or interrupt active sites (for example, phosphorylation sites) resulting in loss-of-function or gain-of-function mutations, or they can occur in such a way that the three-dimensional structure and/or charge balance of the protein is perturbed sufficiently to interfere with normal function. Genomic analysis of large numbers of tumors reveals that mutations often occur at a limited number of amino acid positions. Therefore, a majority of the common mutations can be represented by a relatively small number of potential tumor-associated antigens or T cell epitopes.

A “recurrent cancer mutation” is a change in the amino acid sequence of a protein that occurs in multiple types of cancer and/or in multiple subjects having a particular types of cancer. Such mutations associated with a cancer can result in tumor-associated antigens that are not normally present in corresponding healthy tissue.

Tumor-driver genes and cancer-associated proteins having common mutations that occur across multiple cancers or among multiple cancer patients are known, and sequencing data across multiple tumor samples and multiple tumor types exists. See, e.g., Chang et al. (2016) Nat Biotechnol 34(2):155-163; Tamborero et al. (2013) Sci Rep 3:2650, each of which is herein incorporated by reference in its entirety.

Each antigenic peptide can also be hydrophilic or can score up to or below a certain hydropathy threshold, which can be predictive of secretability in Listeria monocytogenes or another bacteria of interest. For example, antigenic peptides can be scored by a Kyte and Doolittle hydropathy index 21 amino acid window, and all scoring above a cutoff (around 1.6) can be excluded as they are unlikely to be secretable by Listeria monocytogenes. Likewise, the combination of antigenic peptides or the fusion polypeptide can be hydrophilic or can score up to or below a certain hydropathy threshold, which can be predictive of secretability in Listeria monocytogenes or another bacteria of interest.

The antigenic peptides can be linked together in any manner. For example, the antigenic peptides can be fused directly to each other with no intervening sequence. Alternatively, the antigenic peptides can be linked to each other indirectly via one or more linkers, such as peptide linkers. In some cases, some pairs of adjacent antigenic peptides can be fused directly to each other, and other pairs of antigenic peptides can be linked to each other indirectly via one or more linkers. The same linker can be used between each pair of adjacent antigenic peptides, or any number of different linkers can be used between different pairs of adjacent antigenic peptides. In addition, one linker can be used between a pair of adjacent antigenic peptides, or multiple linkers can be used between a pair of adjacent antigenic peptides.

Any suitable sequence can be used for a peptide linker. As an example, a linker sequence may be, for example, from 1 to about 50 amino acids in length. Some linkers may be hydrophilic. The linkers can serve varying purposes. For example, the linkers can serve to increase bacterial secretion, to facilitate antigen processing, to increase flexibility of the fusion polypeptide, to increase rigidity of the fusion polypeptide, or any other purpose. In some cases, different amino acid linker sequences are distributed between the antigenic peptides or different nucleic acids encoding the same amino acid linker sequence are distributed between the antigenic peptides (e.g., SEQ ID NOS: 84-94) in order to minimize repeats. This can also serve to reduce secondary structures, thereby allowing efficient transcription, translation, secretion, maintenance, or stabilization of the nucleic acid (e.g., plasmid) encoding the fusion polypeptide within a Lm recombinant vector strain population. Other suitable peptide linker sequences may be chosen, for example, based on one or more of the following factors: (1) their ability to adopt a flexible extended conformation; (2) their inability to adopt a secondary structure that could interact with functional epitopes on the antigenic peptides; and (3) the lack of hydrophobic or charged residues that might react with the functional epitopes. For example, peptide linker sequences may contain Gly, Asn and Ser residues. Other near neutral amino acids, such as Thr and Ala may also be used in the linker sequence Amino acid sequences which may be usefully employed as linkers include those disclosed in Maratea et al. (1985) Gene 40:39-46; Murphy et al. (1986) Proc Natl Acad Sci USA 83:8258-8262; U.S. Pat. Nos. 4,935,233; and 4,751,180, each of which is herein incorporated by reference in its entirety for all purposes. Specific examples of linkers include those in the Table 2 (each of which can be used by itself as a linker, in a linker comprising repeats of the sequence, or in a linker further comprising one or more of the other sequences in Table 2), although others can also be envisioned (see, e.g., Reddy Chichili et al. (2013) Protein Science 22:153-167, herein incorporated by reference in its entirety for all purposes). Unless specified, “n” represents an undetermined number of repeats in the listed linker.

TABLE 2
Linkers.

| | | SEQ | |
|---|---|---|---|
| Peptide | | ID | Hypothetical |
| Linker | Example | NO: | Purpose |
| (GAS)ₙ | GASGAS | 33 | Flexibility |
| (GSA)ₙ | GSAGSA | 34 | Flexibility |
| (G)ₙ; | GGGG | 35 | Flexibility |
| n = 4-8 | | | |
| (GGGGS)ₙ; | GGGGS | 36 | Flexibility |
| n = 1-3 | | | |
| VGKGGSGG | VGKGGSGG | 37 | Flexibility |
| (PAPAP)ₙ | PAPAP | 38 | Rigidity |
| (EAAAK)ₙ; | EAAAK | 39 | Rigidity |
| n = 1-3 | | | |
| (AYL)ₙ | AYLAYL | 40 | Antigen Processing |
| (LRA)ₙ | LRALRA | 41 | Antigen Processing |
| (RLRA)ₙ | RLRA | 42 | Antigen Processing |

B. PEST-Containing Peptides

The recombinant fusion proteins disclosed herein comprise a PEST-containing peptide. The PEST-containing peptide may at the amino terminal (N-terminal) end of the fusion polypeptide (i.e., N-terminal to the antigenic peptides), may be at the carboxy terminal (C-terminal) end of the fusion polypeptide (i.e., C-terminal to the antigenic peptides), or may be embedded within the antigenic peptides. In some recombinant Listeria strains and methods, a PEST containing peptide is not part of and is separate from the fusion polypeptide. Fusion of an antigenic peptides to a PEST-like sequence, such as an LLO peptide, can enhance the immunogenicity of the antigenic peptides and can increase cell-mediated and antitumor immune responses (i.e., increase cell-mediated and anti-tumor immunity). See, e.g., Singh et al. (2005) J Immunol 175(6):3663-3673, herein incorporated by reference in its entirety for all purposes.

A PEST-containing peptide is one that comprises a PEST sequence or a PEST-like sequence. PEST sequences in eukaryotic proteins have long been identified. For example, proteins containing amino acid sequences that are rich in prolines (P), glutamic acids (E), serines (S) and threonines (T) (PEST), generally, but not always, flanked by clusters containing several positively charged amino acids, have rapid intracellular half-lives (Rogers et al. (1986) Science 234:364-369, herein incorporated by reference in its entirety for all purposes). Further, it has been reported that these sequences target the protein to the ubiquitin-proteasome pathway for degradation (Rechsteiner and Rogers (1996) Trends Biochem. Sci. 21:267-271, herein incorporated by reference in its entirety for all purposes). This pathway is also used by eukaryotic cells to generate immunogenic peptides that bind to MHC class I and it has been hypothesized that PEST sequences are abundant among eukaryotic proteins that give rise to immunogenic peptides (Realini et al. (1994) FEBS Lett. 348:109-113, herein incorporated by reference in its entirety for all purposes). Prokaryotic proteins do not normally contain PEST sequences because they do not have this enzymatic pathway. However, a PEST-like sequence rich in the amino acids proline (P), glutamic acid (E), serine (S) and threonine (T) has been reported at the amino terminus of LLO and has been reported to be essential for L. monocytogenes pathogenicity (Decatur and Portnoy (2000) Science 290:992-995, herein incorporated by reference in its entirety for all purposes). The presence of this PEST-like sequence in LLO targets the protein for destruction by proteolytic machinery of the host cell so that once the LLO has served its function and facilitated the escape of L. monocytogenes from the phagosomal or phagolysosomal vacuole, it is destroyed before it can damage the cells.

Identification of PEST and PEST-like sequences is well-known and is described, for example, in Rogers et al. (1986) Science 234(4774):364-378 and in Rechsteiner and Rogers (1996) Trends Biochem. Sci. 21:267-271, each of which is herein incorporated by reference in its entirety for all purposes. A PEST or PEST-like sequence can be identified using the PEST-find program. For example, a PEST-like sequence can be a region rich in proline (P), glutamic acid (E), serine (S), and threonine (T) residues. Optionally, the PEST-like sequence can be flanked by one or more clusters containing several positively charged amino acids. For example, a PEST-like sequence can be defined as a hydrophilic stretch of at least 12 amino acids in length with a high local concentration of proline (P), aspartate (D), glutamate (E), serine (S), and/or threonine (T) residues. In some cases, a PEST-like sequence contains no positively charged amino acids, namely arginine (R), histidine (H), and lysine (K). Some PEST-like sequences can contain one or more internal phosphorylation sites, and phosphorylation at these sites precedes protein degradation.

In one example, the PEST-like sequence fits an algorithm disclosed in Rogers et al. In another example, the PEST-like sequence fits an algorithm disclosed in Rechsteiner and Rogers. PEST-like sequences can also be identified by an initial scan for positively charged amino acids R, H, and K within the specified protein sequence. All amino acids between the positively charged flanks are counted, and only those motifs containing a number of amino acids equal to or higher than the window-size parameter are considered further. Optionally, a PEST-like sequence must contain at least one P, at least one D or E, and at least one S or T.

The quality of a PEST motif can be refined by means of a scoring parameter based on the local enrichment of critical amino acids as well as the motifs hydrophobicity. Enrichment of D, E, P, S, and T is expressed in mass percent (w/w) and corrected for one equivalent of D or E, one1 of P, and one of S or T. Calculation of hydrophobicity can also follow in principle the method of Kyte and Doolittle (1982) J. Mol. Biol. 157:105, herein incorporated by reference in its entirety for all purposes. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine: Hydropathy index=10*Kyte-Doolittle hydropathy index+45.

A potential PEST motif's hydrophobicity can also be calculated as the sum over the products of mole percent and hydrophobicity index for each amino acid species. The desired PEST score is obtained as combination of local enrichment term and hydrophobicity term as expressed by the following equation:

PEST score=0.55*DEPST−0.5*hydrophobicity index.

Thus, a PEST-containing peptide can refer to a peptide having a score of at least +5 using the above algorithm. Alternatively, it can refer to a peptide having a score of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 35, at least 38, at least 40, or at least 45.

Any other known available methods or algorithms can also be used to identify PEST-like sequences. See, e.g., the CaSPredictor (Garay-Malpartida et al. (2005) Bioinformatics 21 Suppl 1:i169-76, herein incorporated by reference in its entirety for all purposes). Another method that can be used is the following: a PEST index is calculated for each stretch of appropriate length (e.g. a 30-35 amino acid stretch) by assigning a value of one to the amino acids Ser, Thr, Pro, Glu, Asp, Asn, or Gln. The coefficient value (CV) for each of the PEST residues is one and the CV for each of the other AA (non-PEST) is zero.

Examples of PEST-like amino acid sequences are those set forth in SEQ ID NOS: 43-51. One example of a PEST-like sequence is KENSISSMAPPASPPASPKTPIEKKHADEIDK (SEQ ID NO: 43). Another example of a PEST-like sequence is KENSISSMAPPASPPASPK (SEQ ID NO: 44). However, any PEST or PEST-like amino acid sequence can be used. PEST sequence peptides are known and are described, for example, in U.S. Pat. Nos. 7,635,479; 7,665,238; and US 2014/0186387, each of which is herein incorporated by reference in its entirety for all purposes.

The PEST-like sequence can be from a Listeria species, such as from Listeria monocytogenes. For example, the Listeria monocytogenes ActA protein contains at least four such sequences (SEQ ID NOS: 45-48), any of which are suitable for use in the compositions and methods disclosed herein. Other similar PEST-like sequences include SEQ ID NOS: 52-54. Streptolysin O proteins from Streptococcus sp. also contain a PEST sequence. For example, Streptococcus pyogenes streptolysin O comprises the PEST sequence KQNTASTETTTTNEQPK (SEQ ID NO: 49) at amino acids 35-51 and Streptococcus equisimilis streptolysin O comprises the PEST-like sequence KQNTANTETTTTNEQPK (SEQ ID NO: 50) at amino acids 38-54. Another example of a PEST-like sequence is from Listeria seeligeri cytolysin, encoded by the lso gene: RSEVTISPAETPESPPATP (e.g., SEQ ID NO: 51).

Alternatively, the PEST-like sequence can be derived from other prokaryotic organisms. Other prokaryotic organisms wherein PEST-like amino acid sequences would be expected include, for example, other Listeria species.

(I) Listeriolysin O (LLO)

One example of a PEST-containing peptide that can be utilized in the compositions and methods disclosed herein is a listeriolysin O (LLO) peptide. An example of an LLO protein is the protein assigned GenBank Accession No. P13128 (SEQ ID NO: 55; nucleic acid sequence is set forth in GenBank Accession No. X15127). SEQ ID NO: 55 is a proprotein including a signal sequence. The first 25 amino acids of the proprotein is the signal sequence and is cleaved from LLO when it is secreted by the bacterium, thereby resulting in the full-length active LLO protein of 504 amino acids without the signal sequence. An LLO peptide disclosed herein can comprise the signal sequence or can comprise a peptide that does not include the signal sequence. Exemplary LLO proteins that can be used comprise, consist essentially of, or consist of the sequence set forth in SEQ ID NO: 55 or homologues, variants, isoforms, analogs, fragments, fragments of homologues, fragments of variants, fragments of analogs, and fragments of isoforms of SEQ ID NO: 55. Any sequence that encodes a fragment of an LLO protein or a homologue, variant, isoform, analog, fragment of a homologue, fragment of a variant, or fragment of an analog of an LLO protein can be used. A homologous LLO protein can have a sequence identity with a reference LLO protein, for example, of greater than 70%, 72%, 75%, 78%, 80%, 82%, 83%, 85%, 87%, 88%, 90%, 92%, 93%, 95%, 96%, 97%, 98%, or 99%.

Another example of an LLO protein is set forth in SEQ ID NO: 56. LLO proteins that can be used can comprise, consist essentially of, or consist of the sequence set forth in SEQ ID NO: 56 or homologues, variants, isoforms, analogs, fragments, fragments of homologues, fragments of variants, fragments of analogs, and fragments of isoforms of SEQ ID NO: 56.

Another example of an LLO protein is an LLO protein from the Listeria monocytogenes 10403S strain, as set forth in GenBank Accession No.: ZP_01942330 or EBA21833, or as encoded by the nucleic acid sequence as set forth in GenBank Accession No.: NZ_AARZ01000015 or AARZ01000015.1. Another example of an LLO protein is an LLO protein from the Listeria monocytogenes 4b F2365 strain (see, e.g., GenBank Accession No.: YP_012823), EGD-e strain (see, e.g., GenBank Accession No.: NP_463733), or any other strain of Listeria monocytogenes. Yet another example of an LLO protein is an LLO protein from Flavobacteriales bacterium HTCC2170 (see, e.g., GenBank Accession No.: ZP_01106747 or EAR01433, or encoded by GenBank Accession No.: NZ_AAOC01000003). LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

Proteins that are homologous to LLO, or homologues, variants, isoforms, analogs, fragments, fragments of homologues, fragments of variants, fragments of analogs, and fragments of isoforms thereof, can also be used. One such example is alveolysin, which can be found, for example, in Paenibacillus alvei (see, e.g., GenBank Accession No.: P23564 or AAA22224, or encoded by GenBank Accession No.: M62709). Other such homologous proteins are known.

The LLO peptide can be a full-length LLO protein or a truncated LLO protein or LLO fragment. Likewise, the LLO peptide can be one that retains one or more functionalities of a native LLO protein or lacks one or more functionalities of a native LLO protein. For example, the retained LLO functionality can be allowing a bacteria (e.g., Listeria) to escape from a phagosome or phagolysosome, or enhancing the immunogenicity of a peptide to which it is fused. The retained functionality can also be hemolytic function or antigenic function. Alternatively, the LLO peptide can be a non-hemolytic LLO. Other functions of LLO are known, as are methods and assays for evaluating LLO functionality.

An LLO fragment can be a PEST-like sequence or can comprise a PEST-like sequence. LLO fragments can comprise one or more of an internal deletion, a truncation from the C-terminal end, and a truncation from the N-terminal end. In some cases, an LLO fragment can comprise more than one internal deletion. Other LLO peptides can be full-length LLO proteins with one or more mutations.

Some LLO proteins or fragments have reduced hemolytic activity relative to wild type LLO or are non-hemolytic fragments. For example, an LLO protein can be rendered non-hemolytic by deletion or mutation of the activation domain at the carboxy terminus, by deletion or mutation of cysteine 484, or by deletion or mutation at another location.

Other LLO proteins are rendered non-hemolytic by a deletion or mutation of the cholesterol binding domain (CBD) as detailed in U.S. Pat. No. 8,771,702, herein incorporated by reference in its entirety for all purposes. The mutations can comprise, for example, a substitution or a deletion. The entire CBD can be mutated, portions of the CBD can be mutated, or specific residues within the CBD can be mutated. For example, the LLO protein can comprise a mutation of one or more of residues C484, W491, and W492 (e.g., C484, W491, W492, C484 and W491, C484 and W492, W491 and W492, or all three residues) of SEQ ID NO: 55 or corresponding residues when optimally aligned with SEQ ID NO: 55 (e.g., a corresponding cysteine or tryptophan residue). As an example, a mutant LLO protein can be created wherein residues C484, W491, and W492 of LLO are substituted with alanine residues, which will substantially reduce hemolytic activity relative to wild type LLO. The mutant LLO protein with C484A, W491A, and W492A mutations is termed “mutLLO.”

As another example, a mutant LLO protein can be created with an internal deletion comprising the cholesterol-binding domain. The sequence of the cholesterol-binding domain of SEQ ID NO: 55 set forth in SEQ ID NO: 74. For example, the internal deletion can be a 1-11 amino acid deletion, an 11-50 amino acid deletion, or longer. Likewise, the mutated region can be 1-11 amino acids, 11-50 amino acids, or longer (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). For example, a mutated region consisting of residues 470-500, 470-510, or 480-500 of SEQ ID NO: 55 will result in a deleted sequence comprising the CBD (residues 483-493 of SEQ ID NO: 55). However, the mutated region can also be a fragment of the CBD or can overlap with a portion of the CBD. For example, the mutated region can consist of residues 470-490, 480-488, 485-490, 486-488, 490-500, or 486-510 of SEQ ID NO: 55. For example, a fragment of the CBD (residues 484-492) can be replaced with a heterologous sequence, which will substantially reduce hemolytic activity relative to wild type LLO. For example, the CBD (ECTGLAWEWWR; SEQ ID NO: 74) can be replaced with a CTL epitope from the antigen NY-ESO-1 (ESLLMWITQCR; SEQ ID NO: 75), which contains the HLA-A2 restricted epitope 157-165 from NY-ESO-1. The resulting LLO is termed “ctLLO.”

In some mutated LLO proteins, the mutated region can be replaced by a heterologous sequence. For example, the mutated region can be replaced by an equal number of heterologous amino acids, a smaller number of heterologous amino acids, or a larger number of amino acids (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). Other mutated LLO proteins have one or more point mutations (e.g., a point mutation of 1 residue, 2 residues, 3 residues, or more). The mutated residues can be contiguous or not contiguous.

In one example embodiment, an LLO peptide may have a deletion in the signal sequence and a mutation or substitution in the CBD.

Some LLO peptides are N-terminal LLO fragments (i.e., LLO proteins with a C-terminal deletion). Some LLO peptides are at least 494, 489, 492, 493, 500, 505, 510, 515, 520, or 525 amino acids in length or 492-528 amino acids in length. For example, the LLO fragment can consist of about the first 440 or 441 amino acids of an LLO protein (e.g., the first 441 amino acids of SEQ ID NO: 55 or 56, or a corresponding fragment of another LLO protein when optimally aligned with SEQ ID NO: 55 or 56). Other N-terminal LLO fragments can consist of the first 420 amino acids of an LLO protein (e.g., the first 420 amino acids of SEQ ID NO: 55 or 56, or a corresponding fragment of another LLO protein when optimally aligned with SEQ ID NO: 55 or 56). Other N-terminal fragments can consist of about amino acids 20-442 of an LLO protein (e.g., amino acids 20-442 of SEQ ID NO: 55 or 56, or a corresponding fragment of another LLO protein when optimally aligned with SEQ ID NO: 55 or 56). Other N-terminal LLO fragments comprise any ALLO without the activation domain comprising cysteine 484, and in particular without cysteine 484. For example, the N-terminal LLO fragment can correspond to the first 425, 400, 375, 350, 325, 300, 275, 250, 225, 200, 175, 150, 125, 100, 75, 50, or 25 amino acids of an LLO protein (e.g., the first 425, 400, 375, 350, 325, 300, 275, 250, 225, 200, 175, 150, 125, 100, 75, 50, or 25 amino acids of SEQ ID NO: 55 or 56, or a corresponding fragment of another LLO protein when optimally aligned with SEQ ID NO: 55 or 56). In some embodiments, the fragment comprises one or more PEST-like sequences. LLO fragments and truncated LLO proteins can contain residues of a homologous LLO protein that correspond to any one of the above specific amino acid ranges. The residue numbers need not correspond exactly with the residue numbers enumerated above (e.g., if the homologous LLO protein has an insertion or deletion relative to a specific LLO protein disclosed herein). Examples of N-terminal LLO fragments include SEQ ID NOS: 57, 58, and 59. LLO proteins that can be used comprise, consist essentially of, or consist of the sequence set forth in SEQ ID NO: 57, 58, or 59 or homologues, variants, isoforms, analogs, fragments, fragments of homologues, fragments of variants, fragments of analogs, and fragments of isoforms of SEQ ID NO: 57, 58, or 59. In some compositions and methods, the N-terminal LLO fragment set forth in SEQ ID NO: 59 is used. An example of a nucleic acid encoding the N-terminal LLO fragment set forth in SEQ ID NO: 59 is SEQ ID NO: 60.

(2) ActA

Another example of a PEST-containing peptide that can be utilized in the compositions and methods disclosed herein is an ActA peptide. ActA is a surface-associated protein and acts as a scaffold in infected host cells to facilitate the polymerization, assembly, and activation of host actin polymers in order to propel a Listeria monocytogenes through the cytoplasm. Shortly after entry into the mammalian cell cytosol, L. monocytogenes induces the polymerization of host actin filaments and uses the force generated by actin polymerization to move, first intracellularly and then from cell to cell. ActA is responsible for mediating actin nucleation and actin-based motility. The ActA protein provides multiple binding sites for host cytoskeletal components, thereby acting as a scaffold to assemble the cellular actin polymerization machinery. The N-terminus of ActA binds to monomeric actin and acts as a constitutively active nucleation promoting factor by stimulating the intrinsic actin nucleation activity. The actA and hly genes are both members of the 10-kb gene cluster regulated by the transcriptional activator PrfA, and actA is upregulated approximately 226-fold in the mammalian cytosol. Any sequence that encodes an ActA protein or a homologue, variant, isoform, analog, fragment of a homologue, fragment of a variant, or fragment of an analog of an ActA protein can be used. A homologous ActA protein can have a sequence identity with a reference ActA protein, for example, of greater than 70%, 72%, 75%, 78%, 80%, 82%, 83%, 85%, 87%, 88%, 90%, 92%, 93%, 95%, 96%, 97%, 98%, or 99%.

One example of an ActA protein comprises, consists essentially of, or consists of the sequence set forth in SEQ ID NO: 61. Another example of an ActA protein comprises, consists essentially of, or consists of the sequence set forth in SEQ ID NO: 62. The first 29 amino acid of the proprotein corresponding to either of these sequences are the signal sequence and are cleaved from ActA protein when it is secreted by the bacterium. An ActA peptide can comprise the signal sequence (e.g., amino acids 1-29 of SEQ ID NO: 61 or 62), or can comprise a peptide that does not include the signal sequence. Other examples of ActA proteins comprise, consist essentially of, or consist of homologues, variants, isoforms, analogs, fragments, fragments of homologues, fragments of isoforms, or fragments of analogs of SEQ ID NO: 61 or 62.

Another example of an ActA protein is an ActA protein from the Listeria monocytogenes 10403S strain (GenBank Accession No.: DQ054585) the NICPBP 54002 strain (GenBank Accession No.: EU394959), the S3 strain (GenBank Accession No.: EU394960), NCTC 5348 strain (GenBank Accession No.: EU394961), NICPBP 54006 strain (GenBank Accession No.: EU394962), M7 strain (GenBank Accession No.: EU394963), S19 strain (GenBank Accession No.: EU394964), or any other strain of Listeria monocytogenes. LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

ActA peptides can be full-length ActA proteins or truncated ActA proteins or ActA fragments (e.g., N-terminal ActA fragments in which a C-terminal portion is removed). In some embodiments, truncated ActA proteins comprise at least one PEST sequence (e.g., more than one PEST sequence). In addition, truncated ActA proteins can optionally comprise an ActA signal peptide. Examples of PEST-like sequences contained in truncated ActA proteins include SEQ ID NOS: 45-48. Some such truncated ActA proteins comprise at least two of the PEST-like sequences set forth in SEQ ID NOS: 45-48 or homologs thereof, at least three of the PEST-like sequences set forth in SEQ ID NOS: 45-48 or homologs thereof, or all four of the PEST-like sequences set forth in SEQ ID NOS: 45-48 or homologs thereof. Examples of truncated ActA proteins include those comprising, consisting essentially of, or consisting of about residues 30-122, about residues 30-229, about residues 30-332, about residues 30-200, or about residues 30-399 of a full length ActA protein sequence (e.g., SEQ ID NO: 62). Other examples of truncated ActA proteins include those comprising, consisting essentially of, or consisting of about the first 50, 100, 150, 200, 233, 250, 300, 390, 400, or 418 residues of a full length ActA protein sequence (e.g., SEQ ID NO: 62). Other examples of truncated ActA proteins include those comprising, consisting essentially of, or consisting of about residues 200-300 or residues 300-400 of a full length ActA protein sequence (e.g., SEQ ID NO: 62). For example, the truncated ActA consists of the first 390 amino acids of the wild type ActA protein as described in U.S. Pat. No. 7,655,238, herein incorporated by reference in its entirety for all purposes. As another example, the truncated ActA can be an ActA-N100 or a modified version thereof (referred to as ActA-N100*) in which a PEST motif has been deleted and containing the nonconservative QDNKR (SEQ ID NO: 73) substitution as described in US 2014/0186387, herein incorporated by references in its entirety for all purposes. Alternatively, truncated ActA proteins can contain residues of a homologous ActA protein that corresponds to one of the above amino acid ranges or the amino acid ranges of any of the ActA peptides disclosed herein. The residue numbers need not correspond exactly with the residue numbers enumerated herein (e.g., if the homologous ActA protein has an insertion or deletion, relative to an ActA protein utilized herein, then the residue numbers can be adjusted accordingly).

Examples of truncated ActA proteins include, for example, proteins comprising, consisting essentially of, or consisting of the sequence set forth in SEQ ID NO: 63, 64, 65, or 66 or homologues, variants, isoforms, analogs, fragments of variants, fragments of isoforms, or fragments of analogs of SEQ ID NO: 63, 64, 65, or 66. SEQ ID NO: 63 referred to as ActA/PEST1 and consists of amino acids 30-122 of the full length ActA sequence set forth in SEQ ID NO: 62. SEQ ID NO: 64 is referred to as ActA/PEST2 or LA229 and consists of amino acids 30-229 of the full length ActA sequence set forth in the full-length ActA sequence set forth in SEQ ID NO: 62. SEQ ID NO: 65 is referred to as ActA/PEST3 and consists of amino acids 30-332 of the full-length ActA sequence set forth in SEQ ID NO: 62. SEQ ID NO: 66 is referred to as ActA/PEST4 and consists of amino acids 30-399 of the full-length ActA sequence set forth in SEQ ID NO: 62. As a specific example, the truncated ActA protein consisting of the sequence set forth in SEQ ID NO: 64 can be used.

Examples of truncated ActA proteins include, for example, proteins comprising, consisting essentially of, or consisting of the sequence set forth in SEQ ID NO: 67, 69, 70, or 72 or homologues, variants, isoforms, analogs, fragments of variants, fragments of isoforms, or fragments of analogs of SEQ ID NO: 67, 69, 70, or 72. As a specific example, the truncated ActA protein consisting of the sequence set forth in SEQ ID NO: 67 (encoded by the nucleic acid set forth in SEQ ID NO: 68) can be used. As another specific example, the truncated ActA protein consisting of the sequence set forth in SEQ ID NO: 70 (encoded by the nucleic acid set forth in SEQ ID NO: 71) can be used. SEQ ID NO: 71 is the first 1170 nucleotides encoding ActA in the Listeria monocytogenes 10403S strain. In some cases, the ActA fragment can be fused to a heterologous signal peptide. For example, SEQ ID NO: 72 sets forth an ActA fragment fused to an Hly signal peptide.

C. Generating Immunotherapy Constructs Encoding Recombinant Fusion Polypeptides

Also provided herein are methods for generating immunotherapy constructs encoding or compositions comprising the recombinant fusion polypeptides disclosed herein. For example, such methods can comprise selecting and designing antigenic peptides to include in the immunotherapy construct (and, for example, testing the hydropathy of the each antigenic peptide, and modifying or deselecting an antigenic peptide if it scores above a selected hydropathy index threshold value), designing one or more fusion polypeptides comprising each of the selected antigenic peptides, and generating a nucleic acid construct encoding the fusion polypeptide.

The antigenic peptides can be screened for hydrophobicity or hydrophilicity. Antigenic peptides can be selected, for example, if they are hydrophilic or if they score up to or below a certain hydropathy threshold, which can be predictive of secretability in a particular bacteria of interest (e.g., Listeria monocytogenes). For example, antigenic peptides can be scored by Kyte and Doolittle hydropathy index with a 21 amino acid window, all scoring above cutoff (around 1.6) are excluded as they are unlikely to be secretable by Listeria monocytogenes. See, e.g., Kyte-Doolittle (1982) J Mol Biol 157(1):105-132; herein incorporated by reference in its entirety for all purposes. Alternatively, an antigenic peptide scoring about a selected cutoff can be altered (e.g., changing the length of the antigenic peptide). Other sliding window sizes that can be used include, for example, 9, 11, 13, 15, 17, 19, 21, 23, 25, 27 or more amino acids. For example, the sliding window size can be 9-11 amino acids, 11-13 amino acids, 13-15 amino acids, 15-17 amino acids, 17-19 amino acids, 19-21 amino acids, 21-23 amino acids, 23-25 amino acids, or 25-27 amino acids. Other cutoffs that can be used include, for example, the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5, or the cutoff can be 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. The cutoff can vary, for example, depending on the genus or species of the bacteria being used to deliver the fusion polypeptide.

Other suitable hydropathy plots or other appropriate scales include, for example, those reported in Rose et al. (1993) Annu Rev Biomol Struct 22:381-415; Biswas et al. (2003) Journal of Chromatography A 1000:637-655; Eisenberg (1984) Ann Rev Biochem 53:595-623; Abraham and Leo (1987) Proteins: Structure, Function and Genetics 2:130-152; Sweet and Eisenberg (1983) Mol Biol 171:479-488; Bull and Breese (1974) Arch Biochem Biophys 161:665-670; Guy (1985) Biophys J 47:61-70; Miyazawa et al. (1985) Macromolecules 18:534-552; Roseman (1988) J Mol Biol 200:513-522; Wolfenden et al. (1981) Biochemistry 20:849-855; Wilson (1981) Biochem J 199:31-41; Cowan and Whittaker (1990) Peptide Research 3:75-80; Aboderin (1971) Int J Biochem 2:537-544; Eisenberg et al. (1984) J Mol Biol 179:125-142; Hopp and Woods (1981) Proc Natl Acad Sci USA 78:3824-3828; Manavalan and Ponnuswamy (1978) Nature 275:673-674; Black and Mould (1991) Anal Biochem 193:72-82; Fauchere and Pliska (1983) Eur J Med Chem 18:369-375; Janin (1979) Nature 277:491-492; Rao and Argos (1986) Biochim Biophys Acta 869:197-214; Tanford (1962) Am Chem Soc 84:4240-4274; Welling et al. (1985) FEBS Lett 188:215-218; Parker et al. (1986) Biochemistry 25:5425-5431; and Cowan and Whittaker (1990) Peptide Research 3:75-80, each of which is herein incorporated by reference in its entirety for all purposes.

Optionally, the antigenic peptides can be scored for their ability to bind to the subject human leukocyte antigen (HLA) type (for example by using the Immune Epitope Database (IED) available at www.iedb.org, which includes netMHCpan, ANN, SMMPMBEC. SMM, CombLib_Sidney2008, PickPocket, and netMHCcons) and ranked by best MHC binding score from each antigenic peptide. Other sources include TEpredict (tepredict.sourceforge.net/help.html) or other available MHC binding measurement scales. Cutoffs may be different for different expression vectors such as Salmonella.

Optionally, the antigenic peptides can be screened for immunosuppressive epitopes (e.g., T-reg epitopes, IL-10-inducing T helper epitopes, and so forth) to deselect antigenic peptides or to avoid immunosuppressive influences.

Optionally, a predicative algorithm for immunogenicity of the epitopes can be used to screen the antigenic peptides. However, these algorithms are at best 20% accurate in predicting which peptide will generate a T cell response. Alternatively, no screening/predictive algorithms are used. Alternatively, the antigenic peptides can be screened for immunogenicity. For example, this can comprise contacting one or more T cells with an antigenic peptide, and analyzing for an immunogenic T cell response, wherein an immunogenic T cell response identifies the peptide as an immunogenic peptide. This can also comprise using an immunogenic assay to measure secretion of at least one of CD25, CD44, or CD69 or to measure secretion of a cytokine selected from the group comprising IFNγ, TNF-α, IL-1, and IL-2 upon contacting the one or more T cells with the peptide, wherein increased secretion identifies the peptide as comprising one or more T cell epitopes.

The selected antigenic peptides can be arranged into one or more candidate orders for a potential fusion polypeptide. If there are more usable antigenic peptides than can fit into a single plasmid, different antigenic peptides can be assigned priority ranks as needed/desired and/or split up into different fusion polypeptides (e.g., for inclusion in different recombinant Listeria strains). Priority rank can be determined by factors such as relative size, priority of transcription, and/or overall hydrophobicity of the translated polypeptide. The antigenic peptides can be arranged so that they are joined directly together without linkers, or any combination of linkers between any number of pairs of antigenic peptides, as disclosed in more detail elsewhere herein. The number of linear antigenic peptides to be included can be determined based on consideration of the number of constructs needed versus the mutational burden, the efficiency of translation and secretion of multiple epitopes from a single plasmid, and the MOI needed for each bacteria or Lm comprising a plasmid.

The combination of antigenic peptides or the entire fusion polypeptide (i.e., comprising the antigenic peptides and the PEST-containing peptide and any tags) also be scored for hydrophobicity. For example, the entirety of the fused antigenic peptides or the entire fusion polypeptide can be scored for hydropathy by a Kyte and Doolittle hydropathy index with a sliding 21 amino acid window. If any region scores above a cutoff (e.g., around 1.6), the antigenic peptides can be reordered or shuffled within the fusion polypeptide until an acceptable order of antigenic peptides is found (i.e., one in which no region scores above the cutoff). Alternatively, any problematic antigenic peptides can be removed or redesigned to be of a different size. Alternatively or additionally, one or more linkers between antigenic peptides as disclosed elsewhere herein can be added or modified to change the hydrophobicity. As with hydropathy testing for the individual antigenic peptides, other window sizes can be used, or other cutoffs can be used (e.g., depending on the genus or species of the bacteria being used to deliver the fusion polypeptide). In addition, other suitable hydropathy plots or other appropriate scales could be used.

Optionally, the combination of antigenic peptides or the entire fusion polypeptide can be further screened for immunosuppressive epitopes (e.g., T-reg epitopes, IL-10-inducing T helper epitopes, and so forth) to deselect antigenic peptides or to avoid immunosuppressive influences.

A nucleic acid encoding a candidate combination of antigenic peptides or fusion polypeptide can then be designed and optimized. For example, the sequence can be optimized for increased levels of translation, duration of expression, levels of secretion, levels of transcription, and any combination thereof. For example, the increase can be 2-fold to 1000-fold, 2-fold to 500-fold, 2-fold to 100-fold, 2-fold to 50-fold, 2-fold to 20-fold, 2-fold to 10-fold, or 3-fold to 5-fold relative to a control, non-optimized sequence.

For example, the fusion polypeptide or nucleic acid encoding the fusion polypeptide can be optimized for decreased levels of secondary structures possibly formed in the oligonucleotide sequence, or alternatively optimized to prevent attachment of any enzyme that may modify the sequence. Expression in bacterial cells can be hampered, for example, by transcriptional silencing, low mRNA half-life, secondary structure formation, attachment sites of oligonucleotide binding molecules such as repressors and inhibitors, and availability of rare tRNAs pools. The source of many problems in bacterial expressions is found within the original sequence. The optimization of RNAs may include modification of cis acting elements, adaptation of its GC-content, modifying codon bias with respect to non-limiting tRNAs pools of the bacterial cell, and avoiding internal homologous regions. Thus, optimizing a sequence can entail, for example, adjusting regions of very high (>80%) or very low (<30%) GC content. Optimizing a sequence can also entail, for example, avoiding one or more of the following cis-acting sequence motifs: internal TATA-boxes, chi-sites, and ribosomal entry sites; AT-rich or GC-rich sequence stretches; repeat sequences and RNA secondary structures; (cryptic) splice donor and acceptor sites; branch points; or a combination thereof. Optimizing expression can also entail adding sequence elements to flanking regions of a gene and/or elsewhere in the plasmid.

Optimizing a sequence can also entail, for example, adapting the codon usage to the codon bias of host genes (e.g., Listeria monocytogenes genes). For example, the codons below can be used for Listeria monocytogenes.

TABLE 3
Codons

A = GCA
C = TGT
D = GAT
E = GAA
F = TTC
G = GGT
H = CAT
I = ATT
K = AAA
L = TTA
M = ATG
N = AAC
P = CCA
Q = CAA
R = CGT
S = TCT
T = ACA
V = GTT
W = TGG
Y = TAT
STOP = TAA

A nucleic acid encoding a fusion polypeptide can be generated and introduced into a delivery vehicle such as a bacteria strain or Listeria strain. Other delivery vehicles may be suitable for DNA immunotherapy or peptide immunotherapy, such as a vaccinia virus or virus-like particle. Once a plasmid encoding a fusion polypeptide is generated and introduced into a bacteria strain or Listeria strain, the bacteria or Listeria strain can be cultured and characterized to confirm expression and secretion of the fusion polypeptide comprising the antigenic peptides.

### V. Kits

Also provided are kits comprising a reagent utilized in performing a method disclosed herein or kits comprising a composition, tool, or instrument disclosed herein.

For example, such kits can comprise THP-1 cells. Such kits can also comprise one or more of the following: one or more recombinant bacteria or Listeria strains disclosed herein expressing or not expressing a disease-associated antigen, T cells having reactivity to the disease-associated antigen, enriched T cells having reactivity to the disease-associated antigen, one or more peptides comprising the disease-associated antigen, and material necessary for detecting a T cell expressed cytokine, such as IFNγ, and plates and media for culturing the cells. In addition, such kits can additionally comprise an instructional material which describes use of the THP-1 cells, T cells, and/or recombinant bacteria or Listeria strain to perform the methods disclosed herein. Although model kits are described below, the contents of other useful kits will be apparent in light of the present disclosure.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Listing of Embodiments

1. In some embodiments are described methods of assessing potency of a Listeria-based immunotherapeutic, comprising: (a) infecting antigen presenting cells (APCs) with the recombinant Listeria-based immunotherapeutic to provide infected APCs, wherein the recombinant Listeria-based immunotherapeutic expresses a disease-associated antigenic peptide; (b) co-culturing the infected APCs with a population of T cells enriched for T cells having reactivity to the disease-associated antigenic peptide; and (c) determining a cytokine production profile of the T cells, wherein an increase in the cytokine production indicates expression of the disease-associated antigenic peptide in the infected APCs.

2. The methods of embodiment 1, wherein the APCs are THP-1 cells.

3. The methods of any preceding embodiment, wherein step (a) comprises infecting the APCs with the recombinant Listeria-based immunotherapeutic at a multiplicity of infection (MOI) of 1-200.

4. The methods of embodiment 3, wherein the APCs are infected with the recombinant Listeria-based immunotherapeutic at an MOI of about 1, about 2, about 5, about 10, about 20, about 100, or about 200.

5. The methods of any preceding embodiment, wherein infecting the APCs comprises incubating the APCs with the recombinant Listeria-based immunotherapeutic for 0.5-24 hours.

6. The methods of embodiment 5, wherein infecting the APCs comprises incubating the APCs with the recombinant Listeria-based immunotherapeutic for about 1 hour, about 2 hours, about 5 hours, or about 24 hours.

7. The methods of any preceding embodiment, wherein the APCs are washed and cultured for 18-24 hours prior to co-culture with the T cells.

8. The methods of any preceding embodiment, wherein the ratio of APCs to T cells in step (b) is 1:1 to 4:1.

9. The methods of any preceding embodiment, wherein the number of APCs is about 5000 to about 40,000.

10. The methods of any preceding embodiment, wherein the APCs are co-cultured with the T cells for about 18-24 hours.

11. The methods of any preceding embodiment, wherein the APCs are co-cultured with the T cells in the presence of a protein secretion inhibitor, optionally wherein the protein secretion inhibitor is brefeldin A.

12. The method of any preceding embodiment, wherein determining a cytokine expression profile of the T cells comprises measuring the level of interferon gamma (IFNγ) produced by the T cells.

13. The method of embodiment 12, wherein determining a cytokine expression profile of the T cells comprises measuring the level of IFNγ produced by the T cells and secreted into a culture media.

14. The methods of embodiment 12 or 13, wherein IFNγ is detected by enzyme-linked immunosorbent assay (ELISA).

15. The methods of any preceding embodiment, wherein the disease-associated antigenic peptide is a tumor-associated antigen.

16. The methods of any preceding embodiment, wherein the recombinant Listeria-based immunotherapeutic is a Listeria monocytogenes strain.

17. The methods of embodiment 16, wherein the Listeria monocytogenes comprises a nucleic acid comprising a first open reading frame encoding a fusion polypeptide, wherein the fusion polypeptide comprises a PEST-containing peptide fused to the disease-associated antigenic peptide.

18. The methods of embodiment 17, wherein the PEST-containing peptide is listeriolysin O (LLO) or a fragment thereof, and the disease-associated antigenic peptide is a human papillomavirus (HPV) protein E7 or a fragment thereof.

19. The methods of any one of embodiments 17 or 18, wherein the recombinant Listeria-based immunotherapeutic is an attenuated Listeria monocytogenes strain comprising a deletion of or inactivating mutation in prfA, wherein the nucleic acid is in an episomal plasmid and comprises a second open reading frame encoding a D133V PrfA mutant protein.

20. The methods of embodiment 17, wherein the recombinant Listeria-based immunotherapeutic is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO).

21. The methods of embodiment 16 wherein the Listeria monocytogenes strain is ADXS11-001, and the T cell is an HPV-reactive T cell or an HPV-E7-reactive T cell.

22. A method of assessing potency of a Listeria-based immunotherapeutic, comprising: (a) infecting THP-1 cells with a recombinant Listeria-based immunotherapeutic at an MOI of 1-20 for 2 hours to provide infected THP-1 cells, wherein the recombinant Listeria-based immunotherapeutic comprises a live attenuated Listeria monocytogenes strain genetically modified to express a fusion protein of listeriolysin O (LLO) or a fragment thereof and the human papillomavirus (HPV) 16 protein E7 tumor antigen comprising HPV16 protein 17 or a fragment thereof; (b) washing the THP-1 cells and culturing the THP-1 cells for an additional 18-24 hours in the absence of gentamicin; (c) co-culturing the infected THP-1 cells with T cells having reactivity to an HPV 16 E7 antigenic peptide for 18-24 hours; and (d) measuring interferon gamma (IFNγ) production, wherein an increase in IFNγ production indicates expression of the HPV 16 protein E7 tumor antigen or a fraction thereof in the infected THP-1 cells.

23. The method of embodiment 22, wherein the HPV 16 E7 antigenic peptide comprises SEQ ID NO: 101.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

TABLE 4
Description of Sequences.

| SEQ ID NO | Type | Description |
|---|---|---|
| 1 | DNA | SIINFEKL Tag v1 |
| 2 | DNA | SIINFEKL Tag v2 |
| 3 | DNA | SIINFEKL Tag v3 |
| 4 | DNA | SIINFEKL Tag v4 |
| 5 | DNA | SIINFEKL Tag v5 |
| 6 | DNA | SIINFEKL Tag v6 |
| 7 | DNA | SIINFEKL Tag v7 |
| 8 | DNA | SIINFEKL Tag v8 |
| 9 | DNA | SIINFEKL Tag v9 |
| 10 | DNA | SIINFEKL Tag v10 |
| 11 | DNA | SIINFEKL Tag v11 |
| 12 | DNA | SIINFEKL Tag v12 |
| 13 | DNA | SIINFEKL Tag v13 |
| 14 | DNA | SIINFEKL Tag v14 |
| 15 | DNA | SIINFEKL Tag v15 |
| 16 | Protein | SIINFEKL Tag |
| 17 | DNA | 3xFLAG Tag v1 |
| 18 | DNA | 3xFLAG Tag v2 |
| 19 | DNA | 3xFLAG Tag v3 |
| 20 | DNA | 3xFLAG Tag v4 |
| 21 | DNA | 3xFLAG Tag v5 |
| 22 | DNA | 3xFLAG Tag v6 |
| 23 | DNA | 3xFLAG Tag v7 |
| 24 | DNA | 3xFLAG Tag v8 |
| 25 | DNA | 3xFLAG Tag v9 |
| 26 | DNA | 3xFLAG Tag v10 |
| 27 | DNA | 3xFLAG Tag v11 |
| 28 | DNA | 3xFLAG Tag v12 |
| 29 | DNA | 3xFLAG Tag v13 |
| 30 | DNA | 3xFLAG Tag v14 |
| 31 | DNA | 3xFLAG Tag v15 |
| 32 | Protein | 3xFLAG Tag |
| 33 | Protein | Peptide Linker v1 |
| 34 | Protein | Peptide Linker v2 |
| 35 | Protein | Peptide Linker v3 |
| 36 | Protein | Peptide Linker v4 |
| 37 | Protein | Peptide Linker v5 |
| 38 | Protein | Peptide Linker v6 |
| 39 | Protein | Peptide Linker v7 |
| 40 | Protein | Peptide Linker v8 |
| 41 | Protein | Peptide Linker v9 |
| 42 | Protein | Peptide Linker v10 |
| 43 | Protein | PEST-Like Sequence v1 |
| 44 | Protein | PEST-Like Sequence v2 |
| 45 | Protein | PEST-Like Sequence v3 |
| 46 | Protein | PEST-Like Sequence v4 |
| 47 | Protein | PEST-Like Sequence v5 |
| 48 | Protein | PEST-Like Sequence v6 |
| 49 | Protein | PEST-Like Sequence v7 |
| 50 | Protein | PEST-Like Sequence v8 |
| 51 | Protein | PEST-Like Sequence v9 |
| 52 | Protein | PEST-Like Sequence v10 |
| 53 | Protein | PEST-Like Sequence v11 |
| 54 | Protein | PEST-Like Sequence v12 |
| 55 | Protein | LLO Protein v1 |
| 56 | Protein | LLO Protein v2 |
| 57 | Protein | N-Terminal Truncated LLO v1 |
| 58 | Protein | N-Terminal Truncated LLO v2 |
| 59 | Protein | N-Terminal Truncated LLO v3 |
| 60 | DNA | Nucleic Acid Encoding N-Terminal Truncated |
| | | LLO v3 |
| 61 | Protein | ActA Protein v1 |
| 62 | Protein | ActA Protein v2 |
| 63 | Protein | ActA Fragment v1 |
| 64 | Protein | ActA Fragment v2 |
| 65 | Protein | ActA Fragment v3 |
| 66 | Protein | ActA Fragment v4 |
| 67 | Protein | ActA Fragment v5 |
| 68 | DNA | Nucleic Acid Encoding ActA Fragment v5 |
| 69 | Protein | ActA Fragment v6 |
| 70 | Protein | ActA Fragment v7 |
| 71 | DNA | Nucleic Acid Encoding ActA Fragment v7 |
| 72 | Protein | ActA Fragment Fused to Hly Signal Peptide |
| 73 | Protein | ActA Substitution |
| 74 | Protein | Cholesterol-Binding Domain of LLO |
| 75 | Protein | HLA-A2 restricted Epitope from NY-ESO-1 |
| 76 | Protein | Lm Alanine Racemase |
| 77 | Protein | Lm D-Amino Acid Aminotransferase |
| 78 | DNA | Nucleic Acid Encoding Lm Alanine Racemase |
| 79 | DNA | Nucleic Acid Encoding Lm D-Amino Acid |
| | | Aminotransferase |
| 80 | Protein | Wild Type PrfA |
| 81 | DNA | Nucleic Acid Encoding Wild Type PrfA |
| 82 | Protein | D133V PrfA |
| 83 | DNA | Nucleic Acid Encoding D133V PrfA |
| 84 | DNA | 4X Glycine Linker G1 |
| 85 | DNA | 4X Glycine Linker G2 |
| 86 | DNA | 4X Glycine Linker G3 |
| 87 | DNA | 4X Glycine Linker G4 |
| 88 | DNA | 4X Glycine Linker G5 |
| 89 | DNA | 4X Glycine Linker G6 |
| 90 | DNA | 4X Glycine Linker G7 |
| 91 | DNA | 4X Glycine Linker G8 |
| 92 | DNA | 4X Glycine Linker G9 |
| 93 | DNA | 4X Glycine Linker G10 |
| 94 | DNA | 4X Glycine Linker G11 |
| 95 | Protein | Detoxified Listeriolysin O (dtLLO) |
| 96 | Protein | Modified Cholesterol-Binding Domain of |
| | | dtLLO |
| 97 | Protein | LLO Signal Sequence |
| 98 | Protein | ActA Signal Sequence |
| 99 | Protein | Variant FLAG Tag |
| 100 | Protein | 1T 9mer E7 peptide |
| 101 | Protein | 2T 10mer E7 peptide |

### EXAMPLES

### Example 1. Development of an Assay to Measure Monocyte Presentation of an E7 Epitope Expressed by Recombinant Listeria monocytogenes ADXSII-001

Cells: T cells specific for HPV E7 were generated from the PBMC. White blood cells were collected by leukapheresis and PBMC were purified by Ficoll-Paque density gradient centrifugation. T cells were developed and maintained in X-VIVO20™ medium (Lonza, Walkersville Md.).

The THP-1 cell line (American Type Culture Collection) is a monocytic cell line derived from a patient with acute monocytic leukemia. It has an HLA type of A*02:01, A*09, B*05 and DRB1*01, DRB1*15. The cells were maintained in RPMI 1640 containing 10% FBS.

Antigens: Peptide antigens were synthesized by 21′ Century Biochemicals (Marlboro, Mass.) and consisted of 2 peptides from the E7 protein of HPV. One, referred to as 1T (or 9mer) has the sequence YMLDLQPET (SEQ ID NO: 100) and the second peptide, referred to as 2T (or 10mer) has the sequence YMLDLQPETT (SEQ ID NO: 101). The amino acid sequences are from residues 11-20 of the E7 protein.

Analysis Kits and Reagents: Analysis of cytokine concentrations in culture medium used the U-plex assay kits from Meso Scale Discovery. Expression of T cell receptors specific for E7 peptide bound to HLA-A*02:01 was detected using HLA-A*02:01/peptide tetramers from MBL International (Woburn, Mass.).

Test Articles: Lm-based immunotherapeutic was supplied by Advaxis and included product (ADXS11) which expresses human papillomavirus protein E7 fused to truncated Listeriolysin O (tLLO)) under the control of the hly promoter and a control (XFL7) that doesn't express the E7 fusion protein.

Infection of THP-1: The THP-1 cell line was infected with either ADXS11-001 or XFL7 by incubating 10⁶ cells with dilutions of bacteria in a 1 mL volume of RPMI 1640 10% FBS. Incubation with bacteria was at 37° C., 5% CO₂ for the indicated times, then extracellular bacteria were eliminated by washing and incubation in the presence of 50 μg/mL gentamicin for 1 hour at 37° C. After incubation with gentamicin the cells were washed again and resuspended to the desired cell concentration.

For some assays or experiments, the THP-1 cells were exposed to either 1T or 2T peptide.

Assay development: THP-1 is a monocytic cell line derived from an acute monocytic leukemia patient that expresses the HLA allele, A*02:01.

THP-1 were infected as described above and dilutions were prepared. From each dilution 100 μL were spread on each TSA plate. Colonies were counted after 48 hours. From these colony counts, the two bacterial samples that had roughly equivalent CFU/mL of 1.1×10⁷ and 1.6×10⁷ in the dilution were used to infect the THP-1, representing in an MOI of 10. Infection of the THP-1 cell line was confirmed and approximately 25-35% of the cells were infected based upon dilution of 1 million cells. This pattern was repeated in a second experiment and a two-hour incubation with bacteria followed by a one-hour incubation with gentamicin.

TABLE 5
Infection of THP-1 cells with XFL7 and ADXS11

| | |
|---|---|
| Control Listeria - XFL7 | ADXS11 |

CFU in Inoculum

| Dilution of | | | | |
|---|---|---|---|---|
| inoculum | Plate 1 | Plate 2 | Plate 1 | Plate 2 |
| 10⁻³ | TNTC | TNTC | TNTC | TNTC |
| 10⁻⁴ | 103 | 98 | 138 | 135 |
| 10⁻⁵ | 13 | 10 | 14 | 18 |

CFU in Infected THP-1 Cells

| Dilution | Plate 1 | Plate 2 | Plate 1 | Plate 2 |
|---|---|---|---|---|
| 10⁻² | 270 | 267 | 254 | 335 |
| 10⁻³ | 33 | 37 | 27 | 22 |

TNTC = two numerous to count

Generation of enriched E7 epitope specific T cell population. T cells specific for E7 epitopes have been reported in the literature and there are two distinct epitopes that appear to dominate the response restricted by HLA-A*02:01: a 9-amino acid epitope YMLDLQPET (9mer; SEQ ID NO: 100) and a 10-amino acid epitope, YMLDLQPETT (10mer; SEQ ID NO: 101). We generated enriched T cell populations specific to each of these epitopes (see Example 2).

T cell cytokine expression analysis. An initial experiment using THP-1 as antigen presenting cells was performed using a T cell culture containing 6% E7 (11-20) specific T cells based upon staining with HLA-A*02:01/peptide tetramer. THP-1 cells were infected as described using two different concentrations of bacteria and then co-cultured with the T cells. Controls included uninfected THP-1 cells and THP-1 cells incubated with (exposed to) either the 9mer or 10mer peptide for one hour. After an overnight co-culture, medium was collected for analysis of cytokines. Results are shown in Tables 6 and 7.

TABLE 6
Cytokine production following incubation of THP-1 cells with enriched E7 receptor T cells.

| T cells and THP | IFNγ | IL-10 | IL-12p70 | IL-13 | IL-1β | IL-2 | IL-4 | IL-6 | IL-8 | TNF-α |
|---|---|---|---|---|---|---|---|---|---|---|
| Uninfected | 1350.3 | 2.3 | <LLOD | 110.8 | 0.8 | 4.1 | <LLOD | 4.0 | 358.7 | 51.8 |
| 10mer | 17140.7 | 34.0 | 7.4 | 508.4 | 7.0 | 315.2 | 11.9 | 19.6 | 6099.7 | 1192.3 |
| 9mer | 1508.2 | 2.1 | <LLOD | 120.3 | 1.0 | 3.7 | 0.2 | 4.2 | 405.7 | 57.1 |
| XFL7 | 2303.2 | 14.9 | <LLOD | 160.5 | 125.5 | 6.7 | 0.4 | 6.1 | 5290.8 | 1416.5 |
| (MOI = 10) | | | | | | | | | | |
| XFL7 | 2624.7 | 19.3 | <LLOD | 143.9 | 225.1 | 4.9 | 1.2 | 6.4 | 3623.4 | 2817.6 |
| (MOI = 50) | | | | | | | | | | |
| ADXS11-001 | 1930.9 | 10.0 | <LLOD | 138.4 | 114.2 | 5.1 | 0.3 | 5.0 | 5715.0 | 474.6 |
| (MOI = 10) | | | | | | | | | | |
| ADXS11-001 | 2472.7 | 14.9 | 0.8 | 162.7 | 122.7 | 5.9 | 1.3 | 6.1 | 3719.5 | 1523.9 |
| (MOI = 50) | | | | | | | | | | |
| T cells alone | 297.9 | 2.1 | 0.8 | 56.3 | 0.5 | 3.2 | 0.9 | 2.7 | 52.5 | 18.7 |

TABLE 7A
Production of cytokines by THP-1 in the absence of T cells.

| | | | IL- | | | | | | | |
|---|---|---|---|---|---|---|---|---|---|---|
| THP-1 | IFNγ | IL-10 | 12p70 | IL-13 | IL-1β | IL-2 | IL-4 | IL-6 | IL-8 | TNF-α |
| Uninfected | <LLOD | <LLOD | <LLOD | <LLOD | 0.5 | <LLOD | <LLOD | <LLOD | 9.1 | <LLOD |
| 10mer | <LLOD | <LLOD | <LLOD | <LLOD | 0.1 | <LLOD | <LLOD | <LLOD | 8.1 | <LLOD |
| 9mer | <LLOD | <LLOD | <LLOD | <LLOD | 0.0 | <LLOD | <LLOD | <LLOD | 7.3 | <LLOD |
| control Listeria 1× | 14.4 | 18.5 | 0.9 | 2.0 | 88.8 | <LLOD | <LLOD | 0.9 | 5186.7 | 944.0 |
| control listeria 5× | 21.2 | 25.1 | <LLOD | 2.8 | 190.7 | 0.2 | <LLOD | 1.2 | 2670.1 | 2070.0 |
| E7 Listeria 1× | 7.2 | 12.3 | <LLOD | 2.0 | 90.3 | <LLOD | <LLOD | 0.7 | 7263.7 | 285.8 |
| E7 Listeria 5× | 7.6 | 18.2 | <LLOD | 2.1 | 82.5 | <LLOD | <LLOD | 0.8 | 3352.3 | 702.1 |

Control listeria is XFL7
E7 listeria is ADXS11-001

TABLE 7B
Cytokine secretion by THP-1 after infection in the absence of T cells.

| | IFNγ | IL-10 | IL-12p70 | IL-13 | IL-1β | IL-2 | IL-4 | IL-5 | IL-8 | TNF-α |
|---|---|---|---|---|---|---|---|---|---|---|
| alone | 17.1 | 0.1 | <LLOD | <LLOD | <LLOD | 2.7 | 0.5 | <LLOD | 3.0 | 6.8 |
| control THP | 30.6 | 0.5 | <LLOD | 0.4 | 0.0 | 2.6 | 0.3 | 0.1 | 9.5 | 10.4 |
| THP + E7 peptide | 36.7 | 0.4 | 0.3 | <LLOD | 0.2 | 4.6 | 0.6 | <LLOD | 11.8 | 15.5 |
| control Listeria | 60.7 | 2.5 | <LLOD | 1.4 | 4.6 | 4.7 | 0.5 | 0.7 | 218.6 | 31.7 |
| E7 Listeria | 55.5 | 2.2 | <LLOD | 1.5 | 4.0 | 6.5 | 0.3 | 0.4 | 232.2 | 18.4 |

All values are in pg/mL and are mean values from triplicate samples
<LLOD = Below the lower limit of detection
E7 Listeria = ADXS11-001

Infection of THP-1 with Listeria caused stimulation of both IL-8 and TNF-α.

THP-1 presented the 10mer peptide epitope and T cells responded by producing elevated levels of IFNγ, IL-13, IL-2, IL-8 and TNFα relative to T cells co-cultured with control or uninfected THP-1 cells. The infected THP-1 secreted increased amounts of IL-10, IL-8 and TNFα compared to uninfected or peptide incubated THP-1 cells. The lack of response to infected THP-1 in the assay was not a failure of infection as indicated by the cytokine secretion and confirmed by colony counts of diluted cells on TSA plates.

There were several possible explanations for the muted response with THP-1 cells infected with ADSX11: including failure of THP-1 cells to process the 10mer epitope from full length E7 protein, failure of the THP-1 cells to present the processed 10mer E7 peptide on HLA-A*02:01, insufficient incubation of the infected cells to allow synthesis of the E7 protein and accumulation of the MHC/peptide complex on the cell surface, and/or an inhibitory effect of gentamicin. A culture of T cells specific for the 9mer epitope was used as well as a new culture of 10mer specific T cells. The THP-1 cells were infected as before with XFL7 or ADXS11-001 at an MOI of 10. After treatment with gentamicin for one hour, the cells were cultured overnight in the absence of gentamicin. The next day the infected cells were collected and co-cultured with 9mer or 10mer specific T cells. These co-cultures were set up in standard X-VIVO 15 and X-VIVO 15 without gentamicin or phenol red. The co-cultures were incubated overnight before collecting medium for cytokine analysis. The medium was tested for IFNγ concentrations and optical density. The results demonstrated that both the 9mer and 10mer specific T cells recognized the appropriate peptides. In addition, the 10mer specific T cells recognized ADXS11-001 infected THP-1 when there was no gentamicin in the medium. Therefore, the lack of response with ADXS11-001 infected THP-1 cells in Table 6 was likely caused by the presence of gentamicin and insufficient time to process and present the antigen.

Presence of gentamycin in culture medium interferes with antigen presentation by THP-1 cells during Lm infection. Gentamicin in the culture medium inhibits expression of E7 or antigen presentation for Listeria-based infection. The data further show T cells specific for 10-mer peptide recognize E7 presented by THP-1 infected with E7 Listeria and THP-1 infected with control Listeria does stimulate low levels of IFNγ production.

TABLE 8
IFNγ production by T cells co-incubated with THP-1 cells
exposed to 9mer or 10mer, or infected with control bacteria
or ADX11-011 and incubated in the presence of absence of gentamicin.

| | Without gentamicin | | | 50 μg/mL gentamicin | | |
|---|---|---|---|---|---|---|
| | | with 10mer | with 9mer | | with 10mer | with 9mer |
| | THP-1 | specific | specific | THP-1 | specific | specific |
| | alone | T cells | T cells | alone | T cells | T cells |
| THP-1 | 0.063 | 0.068 | 0.067 | 0.060 | 0.062 | 0.069 |
| THP-1 + 9mer | 0.059 | 0.062 | 0.117 | 0.064 | 0.063 | 0.177 |
| THP-1 + 10mer | 0.062 | 0.200 | 0.066 | 0.058 | 0.226 | 0.075 |
| THP-1 + XFL7 | 0.068 | 0.128 | 0.115 | 0.055 | 0.100 | 0.121 |
| THP-1 + ADX11-001 | 0.061 | 0.253 | 0.160 | 0.057 | 0.100 | 0.123 |
| T cells only | | 0.061 | 0.059 | | 0.054 | 0.057 |

To further explore the importance of the overnight incubation of infected THP-1, THP-1 cells infected overnight were compared to THP-1 cells infected for 2 hours. T cells specific for 9mer and 10mer peptides were co-cultured with each of the THP-1 cells and with uninfected or peptide exposed THP-1. As before, medium was collected after an overnight co-culture and cytokines were measured. Data are shown in Tables 9-10.

Both T cell cultures recognized peptide presented by THP-1 cells by producing increased levels of IFNγ. The 10mer specific T cells also secreted IL-2 and TNFα. Only the 10mer specific T cells also recognized ADXS11-001 infected THP-1 as indicated by increased IFNγ in these cultures. THP-1 cells infected overnight stimulated 10-fold more IFNγ production than cells infected for just 2 hours. The 9mer specific T cells did produce IFNγ when cultured with infected THP-1 cells but there was only a modest difference between cells infected with XFL7 and those infected with ADXS11. A proportional increase in IL-6 and TNF-α was observed with increase in the time of infection.

TABLE 9
Co-culture IFNγ Secretion following
stimulation with Listeria Infected THP-1.

| | | anti 9mer T cells | | anti 10mer T cells | |
|---|---|---|---|---|---|
| | Infection | Mean | CV | Mean | CV |
| Control Lm | 2 hour | 1688 | 14 | 496 | 8 |
| E7 (ADXS11-01) | 2 hour | 1402 | 7 | 1123 | 7 |
| Control Lm | 24 hour | 2414 | 11 | 672 | 44 |
| E7 (ADXS11-01) | 24 hour | 2957 | 14 | 12815 | 8 |

An infection time of 24 hours shows 10-fold increase in the levels of IFNγ stimulation

TABLE 10A
Effect of infection time. Cytokine production by
THP-1 alone at 2 h and 24 hours post-infection.

| | IFNγ | IL-10 | IL-12p70 | IL-13 | IL-1β | IL-2 | IL-5 | IL-6 | TNF-α |
|---|---|---|---|---|---|---|---|---|---|
| control | <LLOD | <LLOD | <LLOD | <LLOD | <LLOD | <LLOD | <LLOD | <LLOD | 0 |
| 9mer | <LLOD | <LLOD | <LLOD | <LLOD | <LLOD | <LLOD | <LLOD | <LLOD | 0 |
| 10mer | <LLOD | <LLOD | <LLOD | <LLOD | <LLOD | <LLOD | <LLOD | <LLOD | 0 |
| ADX 2 hr | 2 | 3 | <LLOD | <LLOD | 14 | <LLOD | 0 | 47 | 347 |
| XFL 2 hr | 2 | 2 | <LLOD | 1 | 21 | <LLOD | 0 | 54 | 390 |
| XFL O/N | 5 | 4 | 0 | <LLOD | 120 | 0 | 1 | 197 | 903 |
| ADX O/N | 6 | 3 | 0 | 2 | 78 | 0 | 1 | 350 | 1093 |

ADX = ADXS11-001

TABLE 10B
Effect of infection time. Cytokine production in T cell co-culture.

| | IFNγ | IL-10 | IL-12p70 | IL-13 | IL-1β | IL-2 | IL-5 | IL-6 | TNF-α |
|---|---|---|---|---|---|---|---|---|---|
| 10mer T cells | | | | | | | | | |
| control | 110 | 0 | <LLOD | <LLOD | <LLOD | <LLOD | <LLOD | <LLOD | <LLOD |
| 10mer | 23328 | 12 | <LLOD | 23 | 3 | 452 | 1 | 31 | 460 |
| 9mer | 103 | 0 | <LLOD | <LLOD | <LLOD | <LLOD | <LLOD | <LLOD | <LLOD |
| ADX 2 hr | 1123 | 3 | 1 | 4 | 22 | 3 | 1 | 409 | 641 |
| ADX O/N | 12815 | 6 | 1 | 10 | 80 | 17 | 1 | 508 | 1030 |
| XLF 2 hr | 496 | 4 | 0 | 2 | 30 | 2 | 1 | 282 | 587 |
| XLF O/N | 672 | 11 | <LLOD | <LLOD | 95 | 3 | 0 | 157 | 575 |
| no APC | 106 | <LLOD | <LLOD | <LLOD | <LLOD | 0 | <LLOD | 0 | 4 |
| 9mer T cells | | | | | | | | | |
| control | 121 | <LLOD | <LLOD | <LLOD | <LLOD | 2 | <LLOD | <LLOD | 4 |
| 10mer | 112 | <LLOD | 0 | <LLOD | <LLOD | 3 | <LLOD | 0 | 2 |
| 9mer | 4713 | 1 | 0 | 3 | 0 | 18 | 0 | 13 | 74 |
| ADX 2 hr | 1402 | 3 | 1 | <LLOD | 23 | 11 | 1 | 557 | 825 |
| ADX O/N | 2957 | 3 | 1 | 4 | 83 | 18 | 1 | 530 | 1248 |
| XLF 2 hr | 1688 | 4 | 0 | 4 | 33 | 11 | 1 | 571 | 743 |
| XLF O/N | 2414 | 4 | 1 | 3 | 133 | 18 | 1 | 247 | 892 |
| no APC | 20 | <LLOD | <LLOD | <LLOD | <LLOD | <LLOD | <LLOD | 0 | 2 |
| THP-1 alone | | | | | | | | | |
| control | <LLOD | <LLOD | <LLOD | <LLOD | <LLOD | <LLOD | <LLOD | <LLOD | 0 |
| 10mer | <LLOD | <LLOD | <LLOD | <LLOD | <LLOD | <LLOD | <LLOD | <LLOD | 0 |
| 9mer | <LLOD | <LLOD | <LLOD | <LLOD | <LLOD | <LLOD | <LLOD | <LLOD | 0 |
| ADX 2 hr | 2 | 3 | <LLOD | <LLOD | 14 | <LLOD | 0 | 47 | 347 |
| ADX O/N | 6 | 3 | 0 | 2 | 78 | 0 | 1 | 350 | 1093 |
| XLF 2 hr | 2 | 2 | <LLOD | 1 | 21 | <LLOD | 0 | 54 | 390 |
| XLF O/N | 5 | 4 | 0 | <LLOD | 120 | 0 | 1 | 197 | 903 |

ADX = ADXS11-001

A separate experiment was performed with a range of MOI and 5 hours infection time. In this experiment only IFNγ was measured since this cytokine is only made by the T cells, not THP-1 cells. While there was still recognition of the ADXS11-001 infected THP-1 cells it was not as great as that observed in overnight cultures when compared to the peptide incubated THP-1.

TABLE 11
Recognition of THP-1 Cells infected
with ADXS11-01 or XFL7 for 5 hours.

| | Mean | CV |
|---|---|---|
| Control | 27 | 8 |
| 10mer | 41305 | 1 |
| ADXS1T001 1:40 (MOI 200) | 10124 | 21 |
| ADXS11-01 1:400 (MOI 20) | 6220 | 24 |
| ADXS11-01 1:4000 (MOI 2) | 2791 | 13 |
| XLF 1:5 (MOI 200) | 20 | 7 |
| XLF 1:50 (MOI 20) | 2432 | 9 |
| XLF 1:500 (MOI 2) | 528 | 34 |

Data represent the mean of triplicate samples. IFNγ in pg/mL.

The MOI used for infection was varied over a range of bacterial concentration. In Table 12 the effect of the different MOI on cytokine secretion by THP-1 is shown. Table 13 shows the effect of different MOI on stimulation of T cells. Secretion of IFNγ by the T cells was related to the MOI with higher MOI stimulating higher IFNγ production, though there was significant IFNγ at all MOI.

TABLE 12
Effect of MOI on Cytokine Secretion by THP-1 cells

| | IFNγ | IL-1β | IL-6 | TNF-α |
|---|---|---|---|---|
| THP-1 Control | 2 | 2 | 78 | 260 |
| THP-1 + 10mer | 1 | 0 | 6 | 104 |
| THP-1 + XLF (MOI 100) | 3 | 145 | 144 | 598 |
| THP-1 + XLF (MOI 10) | 2 | 167 | 191 | 380 |
| THP-1 + XLF (MOI 1) | 5 | 235 | 734 | 1561 |
| THP-1 + ADXS11-01 (MOI 200) | 4 | 200 | 542 | 1055 |
| THP-1 + ADXS11-01 (MOI 20) | 4 | 74 | 585 | 1079 |
| THP-1 + ADXS11-01 (MOI 2) | 6 | 39 | 973 | 1649 |

TABLE 13
Effect of MOI on Presentation of E7 to HPV E7 Specific T cells.

| | IFNγ | IL-1β | IL-6 | TNF-α |
|---|---|---|---|---|
| THP-1 Control | 38 | 1 | 89 | 337 |
| THP-1 + 10mer | 65099 | 6 | 1243 | 2309 |
| THP-1 + XLF 1:10 (MOI 100) | 245 | 135 | 112 | 386 |
| THP-1 + XLF 1:100 (MOI 10) | 241 | 166 | 201 | 292 |
| THP-1 + XLF 1:1000 (MOI 1) | 569 | 188 | 892 | 1605 |
| THP-1 + ADXS11-01 1:20 (MOI 200) | 22758 | 166 | 991 | 1063 |
| THP-1 + ADXS11-01 1:200 (MOI 20) | 15271 | 71 | 1624 | 1640 |
| THP-1 + ADXS11-01 1:2000 (MOI 2) | 5396 | 41 | 3333 | 4121 |

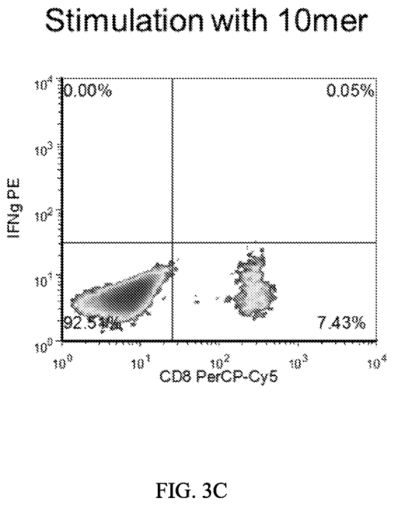

FIGS. 3A-3C show that basal interferon-gamma (IFNγ) was not detectable in THP-1 cells after stimulation with either 9mer or 10mer E7 peptide

Cell number and ratio of T cells to THP-1 cells was also analyzed. The above experiments all used 20,000 T cells and 20,000 THP-1 cells per well (1:1 ratio). We initially investigated the effect of cell number using THP-1 incubated with peptide. The number of both T cells and THP-1 were varied. THP-1 cells ranging from 40,000 cells to 5000 cells per well were incubated with T cells ranging from 20,000 to 5000 cells per well. Effect of varying THP-1 to T cell ratio on IFNγ production was investigated at both higher (1 μg/ml) and lower (0.1 μg/ml) peptide concentrations.

TABLE 14
Effect of cell ratio on IFNγ production at varying peptide concentrations.

| | T cells | | | | | |
|---|---|---|---|---|---|---|
| | 20000 | 10000 | 5000 | 20000 | 10000 | 5000 |
| Peptide conc. | 0.1 μg/ml | | | 1 μg/ml | | |
| THP-1 5000 | 711.40 | 287.77 | 77.56 | 3,178 | 1,513 | 469 |
| THP-1 10000 | 827.31 | 353.46 | 132.50 | 6,859 | 3,060 | 1,321 |
| THP-1 20000 | 2,740.95 | 780.94 | 260.35 | 13,868 | 7,890 | 2,472 |
| THP-1 40000 | 6,158.07 | 2,055.87 | 641.69 | 24,807 | 12,577 | 5,053 |

The level of IFNγ production at the ratio of 40000 THP-1 cells to 10000 T cells was close to the ratio of 20000 THP-1 cells to 20000 T cells. This effect was observed at both low and high peptide concentrations.

We investigated if this effect is reproducible in infected THP cells. Varying concentration of infected THP-1 cells ranging from 40000 to 10000 cells per well were incubated with T cells ranging from 40000 to 10000 cells per well to identify the optimum cell ratio for the assay. The effect of cell ratio on IFNγ production is similar to the effect observed with peptide incubation.

TABLE 15
Effect of cell ratio on IFNγ production at varying MOI

| | | T Cells | | | |
|---|---|---|---|---|---|
| | | THP-1 | | | |
| | MOI | Cells | 10000 | 20000 | 40000 |
| THP-1 + 10mer | — | 40000 | 8078 | 17968 | 34161 |
| ADXS11-01 | 20 | 40000 | 16394 | 36066 | 41832 |
| | 20 | 20000 | 6758 | 17171 | 26187 |
| | 20 | 10000 | 3050 | 7482 | 10382 |
| ADXS11-01 | 2 | 40000 | 8838 | 26505 | 34702 |
| | 2 | 20000 | 3209 | 9986 | 13753 |
| | 2 | 10000 | 1431 | 3011 | 4183 |
| XLF7 | 10 | 40000 | 73.32 | 757.75 | 1470.33 |
| | 10 | 20000 | 51.46 | 757.68 | 1533.00 |

Titration studies with different multiplicity of infection (MOI). THP cells were infected for 2 hours with 3 different dilutions (MOI) of Listeria. Infected or uninfected THP cells were then cultured overnight for 24 hours before co-culturing the T cells. Cells were co-cultured overnight before cytokine analysis.

TABLE 16
Cytokine production by THP-1 cell alone. Consistent with
previous results, THP-1 cells alone produce IL-6 and TNF-α after
infection with Listeria but do not generate IFNγ.

| | IFNγ | IL-1β | IL-6 | TNF-α |
|---|---|---|---|---|
| THP-1 con | <LLOD | <LLOD | 0 | <LLOD |
| THP-1 + 10mer | 9 | 0 | <LLOD | <LLOD |
| THP-1 + XLP 1:100 (MOI = 10) | 6 | 19 | 137 | 552 |
| THP-1 + XLP 1:1000 | 46 | 29 | 346 | 1684 |
| THP-1 + XLP 1:10000 | 2 | 1 | 27 | 99 |
| THP-1 + ADXS11-01 1:100 | 7 | 8 | 130 | 454 |
| (MOI = 10) | | | | |
| THP-1 + ADXS11-01 1:1000 | 13 | 10 | 217 | 713 |
| THP-1 + ADXS11-01 1:10000 | 59 | 1 | 34 | 258 |

TABLE 17
Cytokine Production in THP/T cell co-culture assay. Only IFNγ
production by T cells exhibits proportionality to MOI for infection.

| | IFNγ | IL-1β | IL-6 | TNF-α |
|---|---|---|---|---|
| THP-1 con | 225 | 0 | 1 | 8 |
| THP-1 + 10mer | 33835 | 3 | 23 | 697 |
| THP-1 + XLF 1:100 (MOI = 10) | 969 | 18 | 283 | 576 |
| THP-1 + XLF 1:1000 (MOI = 1) | 812 | 38 | 1157 | 2526 |
| THP-1 + XLF 1:10000 (MOI = 0.1) | 237 | 1 | 177 | 310 |
| THP-1 + ADXS11-01 1:100 | 3879 | 11 | 615 | 681 |
| (MOI = 10) | | | | |
| THP-1 + ADXS11-01 1:1000 | 804 | 15 | 792 | 1242 |
| (MOI = 1) | | | | |
| THP-1 + ADXS11-01 1:10000 | 319 | 5 | 215 | 609 |
| (MOI = 0.1) | | | | |

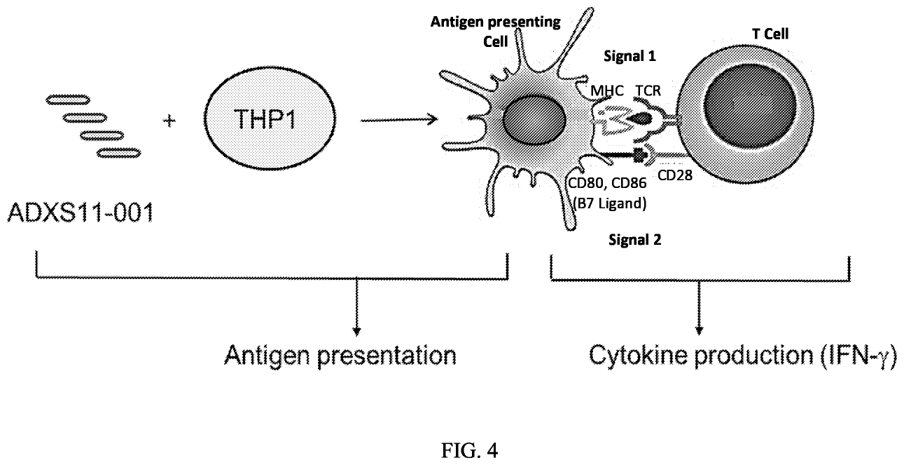

FIG. 4. illustrates the general process for Listeria strain ADXS11-001. For other disease-associated antigens, other Listeria strains are readily substituted.

### Example 2. Generation of Human T Cell Line Having Reactivity to Input Antigen HPV16-E7 (Enriched Antigen Reactive T Cell Population)

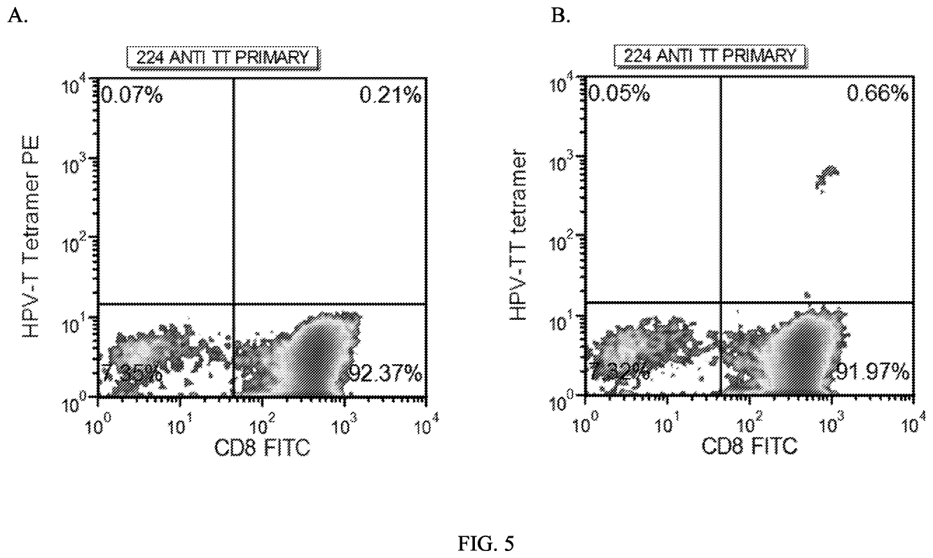

Peripheral blood mononuclear cells (PBMCs) samples were collected from three human HLA-A*02:01 donors: 358, 213, and 224. The peripheral blood mononuclear cells (PBMCs) were cultured with one of two E7 peptides: YMLDLQPET (9mer or 1T; SEQ ID NO: 100) or YMLDLQPETT (10mer or 2T; SEQ ID NO: 101). After 10 days culture, the PBMCs were tested for E7 peptide binding staining and cytokine release. FACS analyses showed that donor 224 contained 0.66% YMLDLQPETT-specific CD8+ T cells (FIG. 5).

TABLE 18
Cytokine release by donor 358 PBMC cultured with peptide.
The donor 358 PBMCs indicated generation of IFNγ
after stimulation 9-mer E7 peptide (SEQ ID NO: 100).

| | |
|---|---|
| stimulated with 1T | stimulated with 2T |
| IFNγ | IFNγ |

| Antigen | Mean | CV | Mean | CV |
|---|---|---|---|---|
| none | 148.3 | 8.1 | 319.5 | 16.4 |
| CMV | 172.3 | 14.5 | 449.8 | 37.5 |
| E7 1T | 1642.7 | 5.8 | 352.4 | 11.8 |
| E7 2T | 183.9 | 15.1 | 359.8 | 24.0 |

TABLE 19
Cytokine release by donor 213 PBMC cultured with 1T peptide.
The donor 213 PBMC indicated generation of interferon-
gamma after stimulation with 9-mer E7 peptide (SEQ ID NO:
100). The responses were higher with 9-mer peptide

IFNγ

| Antigen | Mean | CV |
|---|---|---|
| none | 58.1 | 15.6 |
| CMV | 88.1 | 5.4 |
| E7 1T | 242.6 | 18.1 |
| E7 2T | 86.6 | 36.6 |

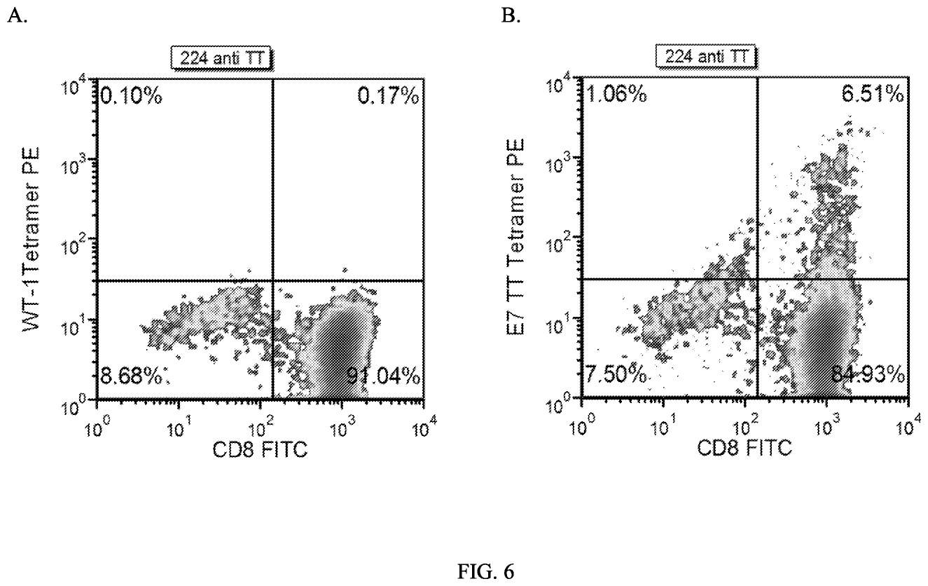

The YMLDLQPETT (SEQ ID NO: 101)-specific CD8+ T cells were cultured, and restimulated with either 2T peptide or a negative control peptide. After re-stimulation, the sample contained 6.51% YMLDLQPETT (SEQ ID NO:101)-specific CD8+ T cells (FIG. 6). CD8 is a marker for cytotoxic T cells. CD8+ cells are cytotoxic T cells. WT is a negative control and, as expected, contains no CD8+/tetramer+ cells.

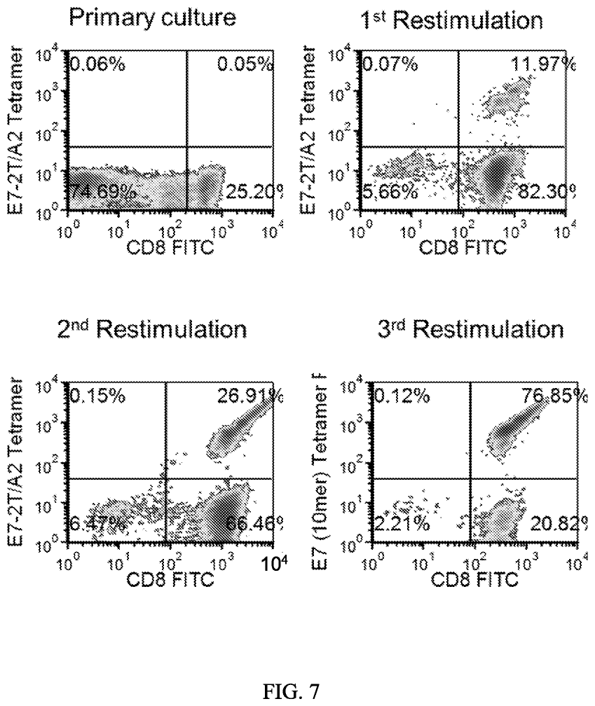

A second round of PMBCs were stimulated and restimulated, to achieve 76.85% YMLDLQPETT (SEQ ID NO:101)-specific CD8+ T cells after 3 rounds of restimulation (FIG. 7).

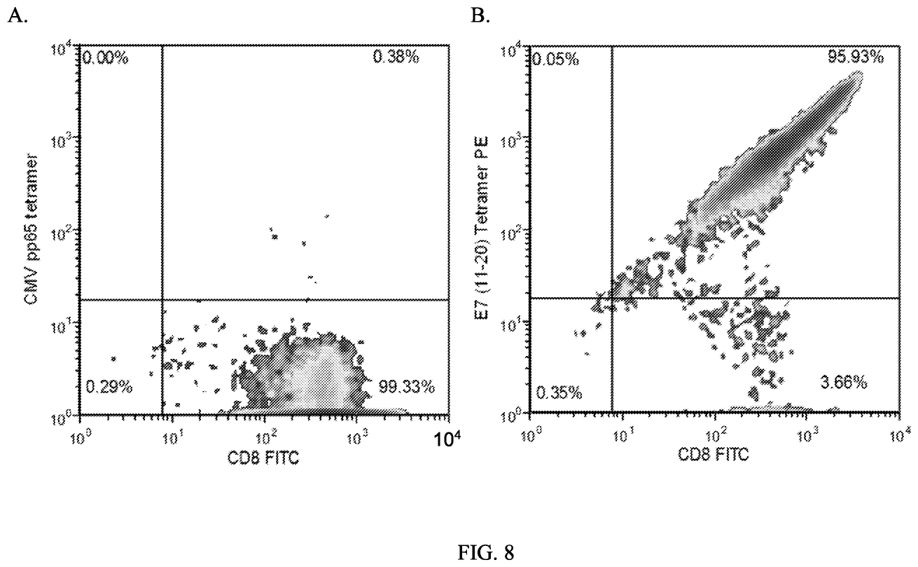

Further rounds of restimulation yielded an enriched T cell population in which 95.93% of the cells were E7 antigen reactive (FIG. 8).

Tetramer staining was used to verify specificity of T cell line for 10mer peptide (YMLDLQPETT; SEQ ID NO: 101). CMV pp65 is a negative control and, as expected, contained little to no CD8+/tetramer+ cells.

To identify cytokines that can be used to measure potency of a Listeria-based therapeutic the following tests were performed. Intracellular cytokine staining was used to detect T cells specificity. THP-1 were incubated with peptide overnight, mixed with T cells from above and centrifuged. The collected cells were then co-cultured at 37° C. for 1 hour. Brefeldin A was then added and the cells were co-cultured for another 4 hours. Brefeldin A inhibits secretion and permits analysis by flow cytometry. Cell were then stained to identify dead cells and CD8+ cells. The cell were then fixed and stained for IFNγ (using fix and permeabilization buffer from eBioscience/Invitrogen).

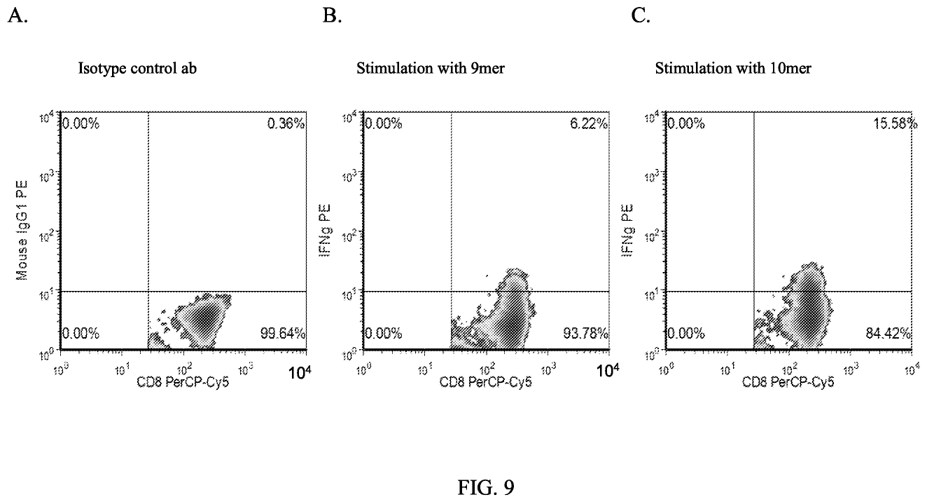

IFNγ was detected in T cells after stimulation with both 9mer and 10mer peptide epitope, T cells generate higher levels of IFNγ after stimulation with 10mer peptide (YMLDLQPETT; SEQ ID NO: 101) when compared to 9mer peptide (YMLDLQPET; SEQ ID NO: 100) (FIG. 9).

The T cell line specific for 10-MER peptide (YMLDLQPETT (SEQ ID NO: 101) was increased significantly in the PBMCs after several rounds of stimulation. The highly specific T cell line was used in the development and optimization of in vitro assay that detects presentation of 10mer peptide.

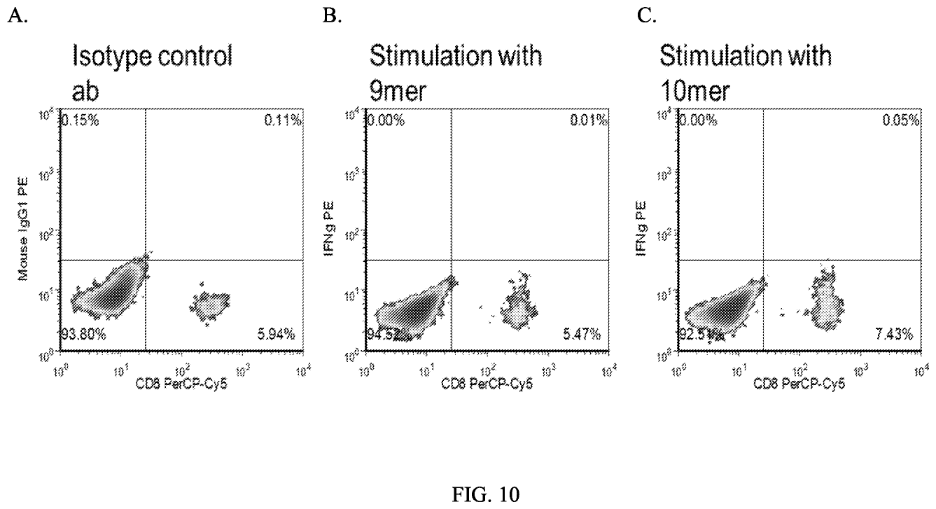

Intracellular cytokine staining of THP-1 cells. Basal IFNγ was not detectable in THP1 cells after stimulation with either 9-mer or 10-mer E7 peptide (FIG. 10).

TABLE 20
Assay set up to detect the secretion of different cytokines
in the presence and absence of T cell line.

| Donor 224 T cells | No T cells |
|---|---|
| No APC | |
| THP-1 | THP-1 |
| THP-1 + E7 peptide | THP-1 + E7 peptide |
| (YMLDLQPETT) | (YMLDLQPETT) |
| THP-1 + control Listeria | THP-1 + control Listeria |
| (XFL7-tLLO) | (XFL7-tLLO) |
| THP-1 + E7 Listeria (ADXS11-01) | THP-1 + E7 Listeria (ADXS11-01) |

TABLE 21
Cytokine secretion by THP-1 after infection (No T cells). Infection
of THP-1 with Listeria caused stimulation of both IL-8 and TNF-α.

| | IFNγ | IL-10 | IL-12p70 | IL-13 | IL-1β | IL-2 | IL-4 | IL-5 | IL-8 | TNF-α |
|---|---|---|---|---|---|---|---|---|---|---|
| alone | 17.1 | 0.1 | <LLOD | <LLOD | <LLOD | 2.7 | 0.5 | <LLOD | 3.0 | 6.8 |
| control THP | 30.6 | 0.5 | <LLOD | 0.4 | 0.0 | 2.6 | 0.3 | 0.1 | 9.5 | 10.4 |
| THP + E7 peptide | 36.7 | 0.4 | 0.3 | <LLOD | 0.2 | 4.6 | 0.6 | <LLOD | 11.8 | 15.5 |
| control Listeria | 60.7 | 2.5 | <LLOD | 1.4 | 4.6 | 4.7 | 0.5 | 0.7 | 218.6 | 31.7 |
| E7 Listeria | 55.5 | 2.2 | <LLOD | 1.5 | 4.0 | 6.5 | 0.3 | 0.4 | 232.2 | 18.4 |

All values are in pg/mL and are mean values from triplicate samples
<LLOD = Below the lower limit of detection

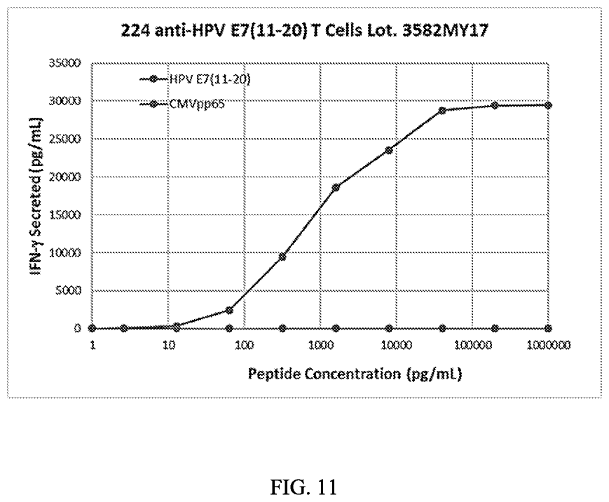

Optimization of infection time required for E7 presentation. THP-1 infected and cultured overnight compared to THP-1 infected for 2 hours and used after gentamicin treatment. Both 9-mer specific T cells and 10-mer specific T cells used. Assays were performed using X-VIVO 15 medium without gentamicin. The following cytokines were measured: IFNγ, IL-1β, IL-2, IL-10, IL-12p70, IL-13, IL-5, IL-6, TNFα. FIG. 11 shows peptide titration with E7 specific T cells.

TABLE 22
IFNγ secretion following stimulation with control
peptide, 1T peptide, or 2T peptide. Antigen reactive
T cell (1T reactive T cells and 2T reactive T cells)
lines showed specific reactivity to the peptide epitope.

| | |
|---|---|
| anti 9mer T cells | anti 10mer T cells |

| | Mean | CV | Mean | CV |
|---|---|---|---|---|
| control | 121 | 47 | 110 | 72 |
| 9mer | 4713 | 7 | 103 | 66 |
| 10mer | 112 | 27 | 23328 | 67 |
| no APC | 20 | 10 | 106 | 9 |

### Example 3. Quantification of Cytokines Secreted by THP1 Cells after Infection with ADXS11-001 or Control Listeria

Flow Diagram for Example of In Vitro Assay

Day 1:
1. THP1 cells actively dividing—Approximately 4 million cells.
2. Infect with ADXS11-001 or Control Listeria (infection time-2 hours).
   a) Positive control: Peptide only (E7 (1T or 2T) peptide).
   b) Negative control: Uninfected cells.
3. Infected and uninfected cells are placed in growth media without gentamycin for 20-24 hours incubated in 6-well plate.

Day 2:
4. Collect and count THP1 cells and coculture with T cells at ratio of 1:1 in 96-well plate, incubate overnight for 18-24 hours.

Day 3:
5. Assay for INFγ in the culture supernatant for each well.

### Example 4. Quantification of Cytokines Secreted by THP1 Cells after Infection with ADXS11-001 or Control Listeria

Flow Diagram for Example of In Vitro Assay

Day 1:
1. Actively dividing THP1 cells. (In some embodiments, approximately 4 million actively dividing THP1 cells.) Infection
2. Infect THP-1 cells with ADXS11-001 or Control Listeria. The ADXS11-001 or Control Listeria are combined with THP-1 cells at an MOI of 1-200. In some embodiments, the ADXS11-001 or Control Listeria are combined with THP-1 cells at an MOI of 1-50, 1-40, 1-30, 1-20, 1-10, or 1-5. In some embodiments, the ADXS11-001 or Control Listeria are combined with THP-1 cells at an MOI of 1, 2, 3, 4, 5, 6, 7, 8, 9, 10, 11, 12, 13, 14, 15, 16, 17, 18, 19, 20, 30, 40, 50, 75, 100, 125, 150, 175, or 200. The ADXS11-001 or Control Listeria are incubated with the THP-1 cells for 0.5-24 hours. In some embodiments, the ADXS11-001 or Control Listeria are incubated with the THP-1 cells for 0.5, 1, 1.5, 2, 3, 4, 5, 6, 7, 8, 9, 10, 12, 14, 16, 18, 20, 22, or 24 hours.
   a) In some embodiments the THP-1 cells are incubated with an E7 peptide as a positive control. The E7 peptide can be, but is not limited to, a 1T (9mer) peptide (SEQ ID NO:100) or 2T (10mer) peptide (SEQ ID NO:101). In some embodiments, uninfected cells are used as a negative control. In some embodiments, Listeria not expressing an E7 antigen or Listeria expressing a different antigen are used as a negative control.
   b) In some embodiments, the extracellular bacteria are removed from the THP-1 cells after the infection step (step 2). The cells can be washed with buffer or growth media. In some embodiments the THP-1 cells are incubated with gentamicin for 0.5 to 2 hours. In some embodiments, the THP-1 cells are incubated with gentamicin for 0.5, 1, 1.5, or 2 hours. In some embodiments, following incubation with gentamicin, the THP-1 cells are washed with buffer or media.
3. After the infection step and optional step 3, in some embodiments, infected and uninfected THP-1 cells are placed in growth media without gentamycin and incubated at 37° C. for 18-24 hours. In some embodiments, infected and uninfected cells are placed in growth media without gentamycin and incubated at 37° C. for 18, 19, 20, 21, 22, 23, or 24 hours. In some embodiments, the cells are incubated in 6-well plates.

Day 2:

Co-Culture with T Cells
4. Infected and uninfected (negative control) THP1 cells are collected and counted. The THP-1 cells are then combined with T cells at ratio of 1:1 to 4:1. In some embodiments the ratio of THP-1 cells to T cells is 1:1, 2:1, 3:1, or 4:1. In some embodiments, 500-40000 THP-1 cells are used. In some embodiments, 5000, 10000, 15000, 20000, 25000, 30000, 35000, or 40000 THP-1 cells are used. In some embodiments, 500-40000 T cells are used. In some embodiments, 5000, 10000, 15000, 20000, 25000, 30000, 35000, or 40000 T cells are used. In some embodiments, the THP-1 cells are co-cultured with the T cells in the presence of a protein secretion inhibitor. In some embodiments, the protein secretion inhibitor is brefeldin A. In some embodiments, the THP-1 cells and T cells are co-cultured in 96-well plates. In some embodiments, the T cells are enriched in E7 peptide-specific T cells. In some embodiments, the T cells are enriched in 1T peptide-specific T cells. In some embodiments, the T cells are enriched in 2T peptide-specific T cells. In some embodiments, the percent of E7 peptide-specific T cells is at least 5%, at least 10%, at least 25%, at least 50%, at least 75%, at least 90%, or at least 95%.

Day 3:

Cytokine Assay
5. Assay for INFγ. In some embodiments, the culture supernatant for each well is assayed for INFγ. In some embodiments, internal IFNγ is measured.

We claim:

1. A method of assessing potency of a Listeria-based immunotherapeutic, comprising:
(a) infecting antigen presenting cells (APCs) with the recombinant Listeria-based immunotherapeutic to provide infected APCs, wherein the recombinant Listeria-based immunotherapeutic expresses a disease-associated antigenic peptide;
(b) co-culturing the infected APCs with a population of T cells enriched for T cells having reactivity to the disease-associated antigenic peptide; and
(c) determining a cytokine production profile of the T cells, wherein an increase in the cytokine production indicates expression of the disease-associated antigenic peptide in the infected APCs.

2. The method of claim 1, wherein the APCs are THP-1 cells.

3. The method of any preceding claim, wherein step (a) comprises infecting the APCs with the recombinant Listeria-based immunotherapeutic at a multiplicity of infection (MOI) of 1-200.

4. The method of claim 3, wherein the APCs are infected with the recombinant Listeria-based immunotherapeutic at an MOI of about 1, about 2, about 5, about 10, about 20, about 100, or about 200.

5. The method of any preceding claim, wherein infecting the APCs comprises incubating the APCs with the recombinant Listeria-based immunotherapeutic for 0.5-24 hours.

6. The method of claim 5, wherein infecting the APCs comprises incubating the APCs with the recombinant Listeria-based immunotherapeutic for about 1 hour, about 2 hours, about 5 hours, or about 24 hours.

7. The method of any preceding claim, wherein the APCs are washed and cultured for 18-24 hours prior to co-culture with the T cells.

8. The method of any preceding claim, wherein the ratio of APCs to T cells in step (b) is 1:1 to 4:1.

9. The method of any preceding claim, wherein the number of APCs in step (b) is about 5000 to about 40,000.

10. The method of any preceding claim, wherein the APCs are co-cultured with the T cells for about 18-24 hours.

11. The method of any preceding claim, wherein the APCs are co-cultured with the T cells in the presence of a protein secretion inhibitor, optionally wherein the protein secretion inhibitor is brefeldin A.

12. The method of any preceding claim, wherein determining a cytokine expression profile of the T cells comprises measuring the level of interferon gamma (IFNγ) produced by the T cells.

13. The method of claim 12, wherein determining a cytokine expression profile of the T cells comprises measuring the level of IFNγ produced by the T cells and secreted into a culture media.

14. The method of claim 12 or 13, wherein IFNγ is detected by enzyme-linked immunosorbent assay (ELISA).

15. The method of any preceding claim, wherein the disease-associated antigenic peptide is a tumor-associated antigen.

16. The method of any preceding claim, wherein the recombinant Listeria-based immunotherapeutic is a Listeria monocytogenes strain.

17. The method of claim 16, wherein the Listeria monocytogenes comprises a nucleic acid comprising a first open reading frame encoding a fusion polypeptide, wherein the fusion polypeptide comprises a PEST-containing peptide fused to the disease-associated antigenic peptide.

18. The method of claim 17, wherein the PEST-containing peptide is listeriolysin O (LLO) or a fragment thereof, and the disease-associated antigenic peptide is a human papillomavirus (HPV) protein E7 or a fragment thereof.

19. The method of claim 17 or 18, wherein the recombinant Listeria-based immunotherapeutic is an attenuated Listeria monocytogenes strain comprising a deletion of or inactivating mutation in prfA, wherein the nucleic acid is in an episomal plasmid and comprises a second open reading frame encoding a D133V PrfA mutant protein.

20. The method of claim 17, wherein the recombinant Listeria-based immunotherapeutic is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of listeriolysin O (LLO).

21. The method of claim 16 wherein the Listeria monocytogenes strain is ADXS11-001, and the T cell is an HPV-reactive T cell or an HPV-E7-reactive T cell.

22. A method of assessing potency of a Listeria-based immunotherapeutic, comprising:
(a) infecting THP-1 cells with a recombinant Listeria-based immunotherapeutic at an MOI of 1-20 for 2 hours to provide infected THP-1 cells, wherein the recombinant Listeria-based immunotherapeutic comprises a live attenuated Listeria monocytogenes strain genetically modified to express a fusion protein of listeriolysin O (LLO) or a fragment thereof and a human papillomavirus (HPV) 16 protein E7 tumor antigen comprising HPV 16 protein E7 or a fragment thereof;
(b) washing the THP-1 cells and culturing the THP-1 cells for an additional 18-24 hours in the absence of gentamicin;
(c) co-culturing the infected THP-1 cells with T cells having reactivity to an HPV16 E7 antigenic peptide for 18-24 hours; and
(d) measuring interferon gamma (IFNγ) production, wherein an increase in IFNγ production indicates expression of the HPV 16 protein E7 tumor antigen or a fragment thereof in the infected THP-1 cells.

23. The method of claim 22, wherein the HPV 16 E7 tumor antigen comprises SEQ ID NO: 101.
